(19) United States
(12) Patent Application Publication
(10) Pub. No.: US 2025/0000901 A1
(43) Pub. Date: Jan. 2, 2025

(54) METHODS AND FOODS INCLUDING SILVER COMPOUNDS TO PROTECT ANIMALS FROM PATHOGENIC BACTERIA AND DISEASE

(71) Applicant: SilverCeuticals Inc., Lindon, UT (US)

(72) Inventors: Keith W. Moeller, Highland, UT (US); Cameron D. Moeller, Pleasant Grove, UT (US); Kirk W. Moeller, American Fork, UT (US); Kory R. Dopp, Eagle Mountain, UT (US); Bracken J. Moeller, Lehi, UT (US); Kimball K. Moeller, American Fork, UT (US); Brennan R. Moeller, Highland, UT (US); Royce A. Wilson, Harrisonville, MO (US)

(21) Appl. No.: 18/755,456

(22) Filed: Jun. 26, 2024

Related U.S. Application Data
(60) Provisional application No. 63/510,466, filed on Jun. 27, 2023.
(60) Provisional application No. 63/652,525, filed on May 28, 2024.

(51) Int. Cl. A61K 33/38; A23K 20/20; A23K 50/70; A61P 1/00; A61P 31/04

(52) U.S. Cl. CPC A61K 33/38; A23K 20/20; A23K 50/70; A61P 1/00; A61P 31/04

(57) ABSTRACT

Methods for using silver compounds to protect the digestive system of animals from pathogenic bacteria and disease are discussed herein. One method includes providing a compound including silver (Ag) particles and facilitating daily oral ingestion of the compound including Ag particles by an animal to protect the digestive system of the animal. Food for treating and/or preventing necrotic enteritis in an animal are also included. One food includes a feed and a compound including Ag particles combined with the feed to create a medicated food for an animal to protect the digestive system of the animal.

### REFERENCE TO RELATED APPLICATIONS

This application claims priority to and the benefits of U.S. Provisional Patent Application No. 63/510,466, filed on Jun. 27, 2023 and U.S. Provisional Patent Application No. 63/652,525, filed on May 28, 2024, the contents of each of which are incorporated herein by reference in their entirety.

### FIELD

The subject matter disclosed herein relates to silver compounds and, more particularly, relates to methods and foods including silver compounds to protect animals (e.g., fowl and other livestock) from pathogenic bacteria and disease.

### BACKGROUND

Animals, including fowls (e.g., chickens, ducks, turkeys, geese, etc.) and at least some other livestock (e.g., pigs, cattle, sheep, goats, horses, etc.), can experience pathogenic bacteria and disease of the digestive system that can be potentially fatal. In fowls, for example, necrotic enteritis (and subclinical necrotic enteritis) is a disease characterized by lesions and/or necrotic tissue in the digestive system (e.g., intestinal epithelium). Necrotic enteritis is caused by intestinal overgrowth of the bacteria Clostridium perfringens Type A and C and can be experienced by juvenile and adult fowl alike. Toxins produced by the bacteria damage the small intestine and liver and can lead to death.

Clinical signs of necrotic enteritis in fowl can include depression characterized by ruffled feathers, diarrhea, dehydration, inappetence, decreased feed conversion, reduced weight gain, and/or weight loss. Necrotic enteritis is rapidly fatal and has a mortality rate of up to 60% in affected birds.

In addition to the digestive system, animals may also experience pathogenic bacteria and disease of the respiratory system. There are many types of respiratory diseases that can be experienced by an animal, some of which can be fatal.

### BRIEF SUMMARY

The subject matter of the present disclosure provides examples of silver compounds and corresponding methods that use silver compounds to protect and/or treat the digestive system of animals, fowls (e.g., chickens, ducks, turkeys, geese, etc.), and/or livestock (e.g., pigs, cattle, sheep, goats, horses, etc.) from pathogenic bacteria and disease. The subject matter of the present disclosure also provides examples of silver compounds and corresponding methods that use silver compounds to protect and/or treat the respiratory system of animals, fowls, and/or livestock from pathogenic bacteria and disease.

In particular, there is a desire to utilize silver compounds to protect, prevent, and/or treat the digestive system of animals, fowls, and other livestock and, particularly, in poultry (e.g., chickens, turkeys, ducks, geese, etc.) from pathogenic bacteria. That is, it is desirable to develop improved methods for using silver compounds to kill the bacteria Clostridium perfringens Type A and C in animals (e.g., fowls, livestock, etc.). It would therefore be desirable to develop improved methods for using silver compounds to prevent livestock, and specifically, fowl from acquiring necrotic enteritis and/or treating necrotic enteritis in affected animals (e.g., fowls, livestock, etc.). Accordingly, the subject matter of the present application has been developed in response to the present state of the art, and in particular, in response to the need to increase the intestinal health of animals and/or decrease the mortality rate of animals infected with necrotic enteritis.

There is a further desire to utilize silver compounds to protect and/or treat the respiratory system of animals from pathogenic bacteria and disease. The silver compounds can kill at least some types of pathogenic bacteria, prevent at least some types of respiratory disease, and/or treat at least some types of respiratory disease in the respiratory system of animals (e.g., fowls, livestock, etc.).

Disclosed herein is a method for protecting animals (e.g., fowl, livestock, and other animals) from pathogenic bacteria and disease. The method includes providing a compound comprising silver (Ag) particles and facilitating daily oral ingestion of the compound by an animal in which providing the compound and facilitating daily oral ingestion of the compound by the animal is performed to protect the digestive system of the animal. The preceding subject matter of this paragraph characterizes example 1 of the present disclosure.

Providing the compound to the animal includes providing a medicated food to the animal in which the medicated food includes the Ag particles and feed and facilitating daily oral ingestion of the compound includes facilitating daily ingestion of the Ag particles via the medicated food. The preceding subject matter of this paragraph characterizes example 2 of the present disclosure, wherein example 2 also includes the subject matter according to example 1, above.

The animal includes a fowl. The preceding subject matter of this paragraph characterizes example 3 of the present disclosure, wherein example 3 also includes the subject matter according to any one of examples 1 or 2, above.

The medicated food includes 0.1 ounces to 1 ounce of the Ag particles per pound. The preceding subject matter of this paragraph characterizes example 4 of the present disclosure, wherein example 4 also includes the subject matter according to any one of examples 1 or 2, above.

The Ag particles include one of nano-silver particles and colloidal silver particles. The preceding subject matter of this paragraph characterizes example 5 of the present disclosure, wherein example 5 also includes the subject matter according to any one of examples 1, 2, or 4 above.

The medicated food comprises a ratio of Ag particles to feed in the range of about 1:10 to about 1:50. The preceding subject matter of this paragraph characterizes example 6 of the present disclosure, wherein example 6 also includes the subject matter according to any one of examples 1 or 2, above.

The Ag particles comprise one of nano-silver particles and colloidal silver particles. The preceding subject matter of this paragraph characterizes example 7 of the present disclosure, wherein example 7 also includes the subject matter according to any one of examples 1, 2, or 6, above.

Facilitating daily ingestion of the Ag particles by the animal includes facilitating daily ingestion of at least 4 ounces of the medicated food. The preceding subject matter of this paragraph characterizes example 8 of the present disclosure, wherein example 8 also includes the subject matter according to any one of examples 1 or 2, above.

Facilitating daily ingestion of the Ag particles by the animal includes facilitating ingestion of the medicated food two times daily. The preceding subject matter of this paragraph characterizes example 9 of the present disclosure, wherein example 9 also includes the subject matter according to any one of examples 1 or 2, above.

Facilitating daily ingestion of the compound two times daily includes facilitating ingestion of 2 ounces of the medicated food two times daily. The preceding subject matter of this paragraph characterizes example 10 of the present disclosure, wherein example 10 also includes the subject matter according to any one of examples 1, 2, or 9, above.

Providing the medicated food to the animal and facilitating daily ingestion of the Ag particles by the animal medicated food is performed to prevent necrotic enteritis. The preceding subject matter of this paragraph characterizes example 11 of the present disclosure, wherein example 11 also includes the subject matter according to any one of examples 1 or 2, above.

Providing the medicated food to the animal and facilitating daily ingestion of the Ag particles by the animal via the medicated food is performed to kill Clostridium perfringens. The preceding subject matter of this paragraph characterizes example 12 of the present disclosure, wherein example 12 also includes the subject matter according to any one of examples 1, 2 or 11, above.

Providing the medicated food to the animal and facilitating daily ingestion of the Ag particles by the animal via the medicated food is performed to kill Clostridium perfringens. The preceding subject matter of this paragraph characterizes example 13 of the present disclosure, wherein example 13 also includes the subject matter according to any one of examples 1 or 2, above.

Disclosed herein is another method for protecting animals (e.g., fowl, livestock, and other animals) from pathogenic bacteria and disease. The method includes facilitating daily oral ingestion of a first compound including Ag particles via a medicated starter food by a juvenile animal and, in response to the juvenile animal maturing into an adult animal, facilitating daily oral ingestion of a second compound including Ag particles via a medicated grower food. The preceding subject matter of this paragraph characterizes example 14 of the present disclosure.

The first compound including Ag particles in the medicated starter food and the second compound including Ag particles in the medicated grower food include one of a same amount of Ag particles by weight or volume or different amounts of Ag particles by weight or volume. The preceding subject matter of this paragraph characterizes example 15 of the present disclosure, wherein example 15 also includes the subject matter according to example 14, above.

A food for at least one of treating or preventing necrotic enteritis in an animal is also disclosed. The food includes a feed and a compound including Ag particles combined with the feed to create a medicated food for the animal in which the medicated food is configured to protect the digestive system of the animal. The preceding subject matter of this paragraph characterizes example 16 of the present disclosure.

The compound including Ag particles incudes 0.5 ppm to 100 ppm of the Ag particles. The preceding subject matter of this paragraph characterizes example 17 of the present disclosure, wherein example 17 also includes the subject matter according to example 16, above.

The Ag particles include one of nano-silver particles and colloidal silver particles. The preceding subject matter of this paragraph characterizes example 18 of the present disclosure, wherein example 18 also includes the subject matter according to example 16, above.

The medicated food includes a ratio of Ag particles to feed in the range of 1:50 to 1:10. The preceding subject matter of this paragraph characterizes example 19 of the present disclosure, wherein example 19 also includes the subject matter according to example 16, above.

The medicated food comprises 0.1 ounces to 1.0 ounce of the Ag particles by weight per pound of the medicated food. The preceding subject matter of this paragraph characterizes example 20 of the present disclosure, wherein example 20 also includes the subject matter according to example 16, above.

### BRIEF DESCRIPTION OF THE DRAWINGS

A more particular description of the embodiments briefly described above will be rendered by reference to specific embodiments that are illustrated in the appended drawings. Understanding that these drawings depict only some embodiments and are not therefore to be considered to be limiting of scope, the embodiments will be described and explained with additional specificity and detail through the use of the accompanying drawings, in which:

FIG. 1 is a schematic diagram of one embodiment of a silver particle that can protect the digestive system of an animal (e.g., fowls, livestock, etc.) from the bacteria Clostridium perfringens, prevent necrotic enteritis in the animal, and/or treat necrotic enteritis in the animal and/or that can protect the respiratory system of the animal, prevent at least some respiratory diseases in the animal, and/or treat at least some respiratory diseases in animals;

FIG. 2 is a schematic diagram of one embodiment of a molecule of coating for the silver particle illustrated in FIG. 1;

FIGS. 3 through 5 are schematic flow diagrams of various embodiments of a method for using a silver compound in water to protect the digestive system of an animal (fowls, livestock, etc.);

FIG. 6 is a table illustrating mortality and removal weights for chickens (e.g., Cobb 500 Broiler Males) that are 0-24 days old;

FIG. 7 is a table illustrating mortality and removal weights for chickens (e.g., Cobb 500 Broiler Males) that are 0-24 days old summarized by treatment;

FIG. 8 is a table illustrating feed added and removed for chickens (e.g., Cobb 500 Broiler Males) that are 0-24 days old;

FIG. 9 is a table illustrating feed added and removed for chickens (e.g., Cobb 500 Broiler Males) that are 0-24 days old summarized by treatment;

FIG. 10 is a table illustrating Day 0 pen weights of chickens (e.g., Cobb 500 Broiler males);

FIG. 11 is a table illustrating Day 17 pen weights of chickens (e.g., Cobb 500 Broiler males);

FIG. 12 is a table illustrating Day 24 pen weights of chickens (e.g., Cobb 500 Broiler males);

FIG. 13 is a table illustrating performance data for days 0-17 of chickens (e.g., Cobb 500 Broiler males);

FIG. 14 is a table illustrating performance data for days 0-17 of chickens (e.g., Cobb 500 Broiler males) summarized by treatment;

FIG. 15 is a table illustrating performance data for days 0-24 of chickens (e.g., Cobb 500 Broiler males);

FIG. 16 is a table illustrating performance data for days 0-24 of chickens (e.g., Cobb 500 Broiler males) summarized by treatment;

FIG. 17 is a table illustrating performance data for days 17-24 of chickens (e.g., Cobb 500 Broiler males);

FIG. 18 is a table illustrating performance data for days 17-24 of chickens (e.g., Cobb 500 Broiler males) summarized by treatment;

FIG. 19 is a table illustrating day 24 average necrotic enteritis lesion scores for chickens (e.g., Cobb 500 Broiler males);

FIG. 20 is a table illustrating day 24 average necrotic enteritis lesion scores for chickens (e.g., Cobb 500 Broiler males) summarized by treatment;

FIG. 21 is a graph illustrating performance data for days 0-17 of chickens (e.g., Cobb 500 Broiler males) summarized by treatment;

FIG. 22 is a graph illustrating performance data for days 0-24 of chickens (e.g., Cobb 500 Broiler males) summarized by treatment;

FIG. 23 is a graph illustrating performance data for days 17-24 summarized by treatment;

FIG. 24 is a graph illustrating average necrotic enteritis lesion scores of chickens (e.g., Cobb 500 Broiler males) summarized by treatment;

FIGS. 25 through 27 are schematic flow diagrams of various embodiments of a method for using a silver compound to protect the respiratory system of an animal (e.g., fowl, livestock, or other animal);

FIG. 28A is a diagram illustrating one embodiment of combining a compound including silver particles with a feed to create a medicated food for animals (e.g., fowl, livestock, or other animal);

FIG. 28B is a diagram illustrating one embodiment of combining a compound including silver particles with a feed to create a medicated starter food for juvenile animals (e.g., juvenile fowl, juvenile livestock, etc.);

FIG. 28C is a diagram illustrating one embodiment of combining a compound including silver particles with a feed to create a medicated grower food for adult animals (e.g., adult fowl, adult livestock, etc.);

FIG. 29 is a chart illustrating one embodiment of a feeding schedule for a group of fowl infected with the bacteria Clostridium perfringens as the fowl grew to determine the amount of silver particles that is to be added to feed to create a medicated food with an amount of silver particles sufficient to kill the Clostridium perfringens, prevent necrotic enteritis in the chicks, and/or treat necrotic enteritis in the fowl as they aged from juvenile to adulthood;

FIGS. 30 through 32 are schematic flow diagrams of various embodiments of a method for using a medicated food including silver particles to protect the digestive system of an animal (e.g., fowl, livestock, or other animal);

FIGS. 33 through 35 are schematic flow diagrams of various embodiments of a method for using a medicated starter food including silver particles to protect the digestive system of a juvenile animal (e.g., a juvenile fowl, juvenile livestock, etc.);

FIGS. 36 through 38 are schematic flow diagrams of various embodiments of a method for using a medicated grower food including silver particles to protect the digestive system of an adult animal (e.g., adult fowl, adult livestock, etc.);

FIG. 39 is a schematic flow diagram illustrating one embodiment of a method for using a medicated starter food including silver particles for an animal (e.g., fowl, livestock, or other animal) as a juvenile and a non-medicated grower food for the animal as an adult;

FIG. 40 is a schematic flow diagram illustrating one embodiment of a method for using a non-medicated starter food for an animal (e.g., fowl, livestock, or other animal) as a juvenile and a medicated grower food including silver particles for the animal as an adult; and

FIG. 41 is a schematic flow diagram illustrating one embodiment of a method for using a medicated starter food including silver particles and a medicated grower food including silver particles to protect the digestive system of an animal (e.g., fowl, livestock, or other animal) as the animal grows from a juvenile to an adult.

### DETAILED DESCRIPTION

The various embodiments disclosed herein provide silver compounds and methods that can protect the digestive system and/or respiratory system of fowl, livestock, and other animals. The silver compounds include silver particles that, when ingested by, for example, a fowl (e.g., a chicken, a turkey, a duck, a goose, etc.), livestock (e.g., pigs, cattle, sheep, goats, horses, etc.), or other animal(s), can protect the animal's digestive system from the bacteria Clostridium perfringens (and/or other pathogenic bacteria), prevent necrotic enteritis, and/or treat necrotic enteritis in the animal. Further, the silver compounds include silver particles that, when inhaled by an animal, can protect the animal's respiratory system from the pathogenic bacteria, prevent respiratory disease, and/or treat respiratory disease in the animal.

As disclosed herein, “silver particles” can include, but are not limited to, “nano-silver particles,” “nano-silver,” “colloidal silver particles,” “colloidal silver,” “ionic silver particles,” “ionic silver,” “solution silver particles,” “solution silver,” “suspension silver particles,” and “suspension silver,” among other particles that can include silver that are possible and contemplated herein. In some embodiments, “silver particles” refers to particles that, in whole or in part, comprise silver in an aqueous medium or solution.

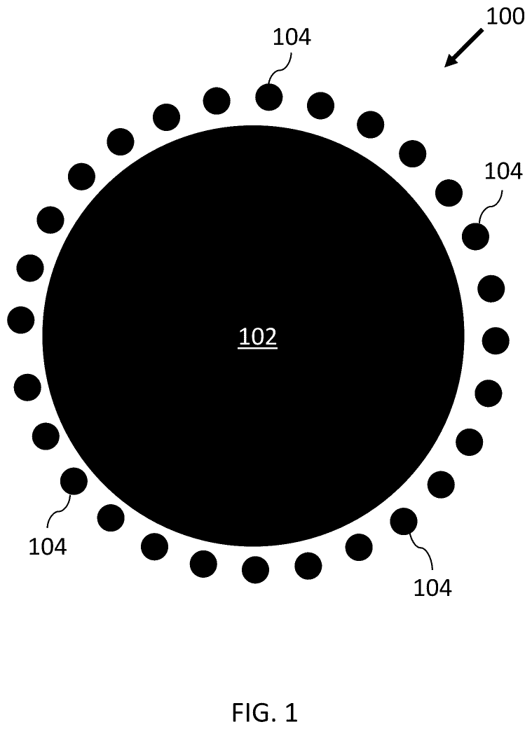

With reference to the drawings, FIG. 1 is a diagram of one embodiment of a silver particle 100, which can also be referred to herein as, an Ag particle. The silver particle 100 can protect the digestive system and/or respiratory system of a fowl or other livestock. The silver particle 100, when ingested by an animal (e.g., fowls, livestock, etc.) can protect the animal's digestive system from the bacteria Clostridium perfringens (and/or other pathogenic bacteria), prevent necrotic enteritis, and/or treat necrotic enteritis in the animal. Further, the silver particles 100, when inhaled by an animal, can protect the animal's respiratory system from the pathogenic bacteria, prevent respiratory disease, and/or treat respiratory disease in the animal.

A silver particle 100 may be characterized in terms of valence of the silver included therein. In the exemplary embodiment shown in FIG. 1, the silver particle 100 includes a silver core 102 and a silver coating 104.

A silver core 102, in various embodiments, includes metallic silver (Ag(0)) to form a metallic silver core. In additional embodiments, the silver core 102 includes metallic nano-silver to form a metallic nano-silver core.

In various embodiments, the silver coating 104 comprises Ag (I), Ag (II), or Ag (III) in an ionic oxidation state. In some embodiments, the silver coating 104 includes silver oxide (e.g., AgO). That is, the silver particle 100 comprises Ag(0) and AgO, where AgO is present as a coating on the silver core 102 (e.g., a metallic nano-silver core).

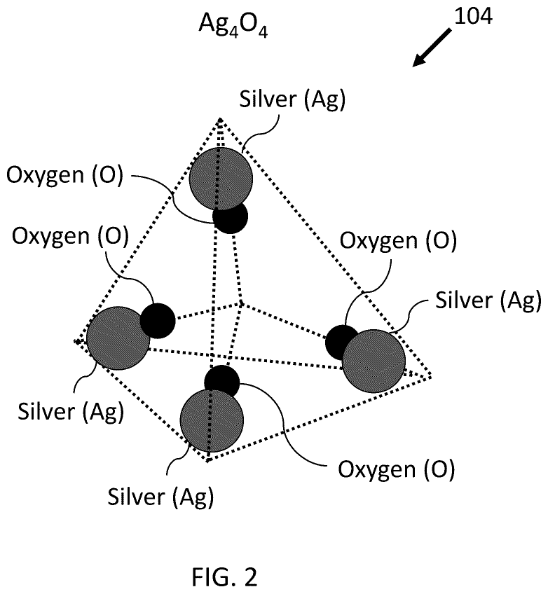

In other embodiments (see, FIG. 2), the silver coating 104 may be at least partially in the form of silver II oxide (Ag₄O₄). In a molecule of this material, two of the silver atoms are in the 1+ state (silver I) and the other two silver molecules are in the 3+ state (silver III). Under certain conditions, these molecules can give rise to silver atoms (Ag) in the 2+ (silver II) state attached to respective oxygen atoms (O). Thus, the present disclosure provides silver particles comprising a metallic silver core 102 and silver coating 104 of silver oxide, the silver oxide being selected from AgO and Ag₄O₄.

The purity of a silver particle 100 may be any suitable percentage of silver purity that is capable of disinfecting a surface. In various embodiments, the percentage of silver purity of a silver particle 100 is in the range of about 50% to about 99.9999%, among other percentages of silver purity that are greater than 99.9999% or less than 50% that are possible and contemplated herein. In certain embodiments, the percentage of silver purity of a silver particle 100 is about 99.99%, among other percentages of silver purity that are greater than or less than about 99.99% that are possible and contemplated herein.

In various embodiments, a plurality of silver particles 100 are added to and/or mixed with one or more liquids and/or solutions to form a silver compound and/or silver composition that can be used to protect and/or treat the digestive system of animals from pathogenic bacteria and disease. In certain embodiments, the silver compound and/or silver composition can be used to prevent Clostridium perfringens from affecting an animal (e.g., fowl, livestock, or other animal) and/or prevent necrotic enteritis from developing in the animal. In additional or alternative embodiments, the silver compound and/or silver composition can be used to kill Clostridium perfringens in the animal and/or treat necrotic enteritis in the animal.

In various embodiments, one liquid includes water (H₂O) and/or other aqueous solution. In some embodiments, the water or aqueous solution includes pure water and/or purified water.

An aqueous compound comprising silver particles 100 may be prepared using any suitable method and/or technique that is known or developed in the future. For example, the preparation of a compound comprising silver particles 100 may utilize an electrochemical cell comprising electrodes. One process includes: (a) placing a silver electrode in contact with a quantity of high purity water; (b) conveying electrical current through the silver electrode to thereby separate particles of silver from the silver electrode in a manner sufficient to cause production of suspended silver particles within the water; and (c) agitating the water during said production of suspended silver particles to thereby disperse the silver particles into a more uniform concentration within the water such that a higher quantity of silver particles can be produced per batch.

Another example technique for preparing a compound comprising silver particles 100 includes: (a) establishing an electrical circuit comprising a current source, and a first conductor electrically connected to the current source and a second conductor electrically connected to the current source in which the first conductor is spaced apart from the second conductor, and at least one of the conductors is made of elemental silver; (b) closing the circuit by placing the first conductor and the second conductor in communication with a fluidic resistor; (c) operating the current source to supply alternating current simultaneously to the first conductor and the second conductor such that voltage is increasing and decreasing within the first and second conductors in alternating tandem to thereby cause silver particles to separate from the first electrode and enter the fluidic resistor and become disposed in suspension within the fluidic resistor; and (d) selectively adjusting the electrodes by moving them toward the fluidic resistor to compensate for decrease in electrode length due to gradual separation of silver particles therefrom to thereby prevent arcing from occurring between the electrodes and said fluidic resistor.

In various embodiments, the total amount of silver particles 100 in a silver compound that can protect the digestive system of an animal (e.g., fowls, livestock, etc.) from Clostridium perfringens and/or treat necrotic enteritis in the animal is in the range of about 0.5 parts per million (0.5 ppm), by weight and/or volume, to about 100 parts per million (100 ppm), by weight and/or volume, among other quantities of silver particles 100 that are less than 0.5 ppm and greater than 100 ppm, by weight and/or volume, that are possible, each of which is contemplated herein. In some embodiments, the total amount of silver particles in an Ag compound is about 10 ppm, by weight and/or volume, among other amounts of silver particles 100 greater than or less that about 10 ppm, by weight and/or volume, that are possible, each of which is contemplated herein. In other embodiments, the total amount of silver particles in an Ag compound is about 32 ppm, by weight and/or volume, among other amounts of silver particles 100 greater than or less that about 32 ppm, by weight and/or volume, that are possible, each of which is contemplated herein.

As the silver particles 100 become smaller, a given concentration of silver particles 100 will represent a larger quantity of particles 100. In addition, the total surface area for a given particle concentration will increase. Therefore, particle size and/or range of particle size for the silver particles 100 may further characterize the compounds of the various embodiments, which may include any suitable particle size and/or range of particle size that is/are known or developed in the future capable of protecting the digestive system of an animal from Clostridium perfringens and/or treat necrotic enteritis in the animal.

In some embodiments, a silver compound includes 50-100% by weight and/or volume of the silver particles 100 including a maximum dimension that is less than about 1.0 micrometer (1000 nanometers (nm)), among other percentages and sizes that are possible, each of which is contemplated herein. In certain embodiments, a silver compound includes 50-100% by weight and/or volume of the silver particles 100 including a dimension in the range of about 0.005 micrometers (5 nm) to about 0.01 micrometers (10 nm), among other percentages and sizes that are possible, each of which is contemplated herein. In still other embodiments, a silver compound comprises 50-100% by weight and/or volume of the silver particles 100 including a minimum dimension greater than or equal to about 0.0005 micrometers (0.5 nm), among other percentages and sizes that are possible, each of which is contemplated herein. Here, these silver particles 100 can be considered colloidal silver particles.

In further embodiments, a silver compound comprises 50-100% by weight and/or volume of the silver particles 100 including a maximum dimension that is less than about 0.001 micrometers (1 nm), among other percentages and sizes that are possible, each of which is contemplated herein. Here, these silver particles 100 can be considered ionic silver particles or solution silver particles.

In still further embodiments, a silver compound comprises 50-100% by weight and/or volume of the silver particles 100 including a minimum dimension that is greater than about 1.0 micrometer (1000 nm), among other percentages and sizes that are possible, each of which is contemplated herein. Here, these silver particles 100 can be considered suspension silver particles.

In one embodiment, the silver particles 100 are stable in essentially and/or substantially pure water that is free of, for example, surfactants, etc., among other natural impurities and/or unnatural impurities that are possible and contemplated herein. Additionally, or in another embodiment, the compound of silver particles 100 is essentially and/or substantially colorless.

In various embodiments, the silver compound forms a silver sol. A silver sol, in various embodiments, includes a stable dispersion of solid silver particles 100 homogeneously dispersed in a liquid. In certain embodiments, the silver sol includes a stable dispersion of solid silver particles 100 homogeneously dispersed in water (e.g., pure water) and/or an aqueous solution.

Testing of the various embodiments of a silver compound disclosed herein shows that ingesting (e.g., oral ingestion) the various embodiments of a silver compound disclosed herein (e.g., on a daily basis in drinking water and/or in food) can kill the bacteria Clostridium perfringens in an animal (e.g., fowl, livestock, or other animal). In addition, testing of the various embodiments of a silver compound disclosed herein shows that ingesting (e.g., oral ingestion) the various embodiments of a silver compound disclosed herein (e.g., on a daily basis in drinking water and/or in food) can treat necrotic enteritis in an animal.

In addition to killing the bacteria Clostridium perfringens in the digestive system an animal (e.g., fowl, livestock, or other animal) and/or treating necrotic enteritis in an animal, various tests on fowl show at least a 5% improvement/increase in weight gain, a reduced amount of gut lesions, elimination of gut lesions in the gut, a reduced mortality rate, a zero-mortality rate, and/or an improved feed efficiency in a challenged control group of fowl. Some studies showed that 4 oz Ag compound/gallon of drinking water is helpful in killing the bacteria Clostridium perfringens in the digestive system a fowl and/or treating necrotic enteritis in a fowl to obtain some of the above benefits; however, at least 6 oz Ag compound/gallon of drinking water was used to achieve the desired weight gain improvements over challenged control group and the zero mortality. An unexpected result found that a dosage at 6 oz Ag compound/gallon of drinking water, fowl seemed to experience an improved feed efficiency over even a non-challenged control group, meaning the challenged control group still gained good amount of weight, but ate less feed.

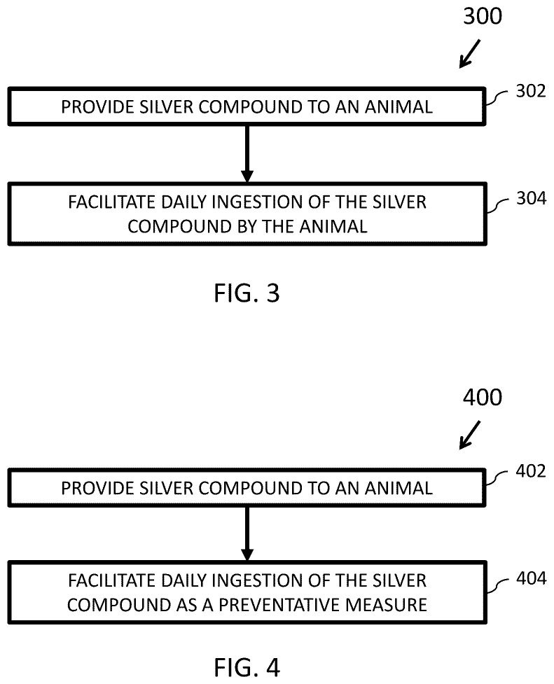

With reference to FIG. 3, FIG. 3 is a block diagram of one embodiment of a method 300 for using a silver compound to protect the digestive system of an animal (e.g., fowl, livestock, or other animal) from the bacteria Clostridium perfringens, prevent necrotic enteritis in the animal, and/or treat necrotic enteritis in the animal. At least in the illustrated embodiment, the method 300 can begin by a providing a silver compound to the animal (block 302).

The silver compound can be provided to the animal in any suitable manner that is known or developed in the future. Examples of providing the silver compound to the animal can include, but is not limited to, automatically and/or passively (e.g., via drinking water) and/or manual administration by a caregiver (e.g., via injection, nebulization, fogging, etc.), among other techniques/methods that are possible, each of which is contemplated herein.

The silver compound can include any embodiment of the silver particles 100 discussed herein. That is, the silver particles 100 may include nano-silver particles 100, colloidal silver particles 100, ionic silver particles 100, solution silver particles 100, and/or suspension silver particles 100.

In some embodiments, the silver compound includes an amount of silver particles 100 (e.g., Ag particles), which can include any amount of silver particles 100 in the range of about 0.5 ppm to about 100 ppm, by weight and/or volume, as discussed elsewhere herein. In certain embodiments, the amount of silver particles 100 is 10 ppm or 32 ppm, by weight and/or volume, among other amounts that are possible, each of which is contemplated herein.

The method 300 further includes facilitating daily ingestion of the silver compound including the silver particles 100 by the animal (block 304). Facilitating daily ingestion of the compound including the silver particles 100 may include any suitable technique and/or method that encourages and/or causes an animal to ingest the silver compound including the silver particles 100 on a daily and/or other similar regular basis. Examples of providing the silver compound to the animal can include, but is not limited to, automatically and/or passively (e.g., via drinking water) and/or manual administration by a caregiver (e.g., via injection, nebulization, fogging, etc.), among other techniques/methods that are possible, each of which is contemplated herein.

The silver compound including the silver particles 100 may be ingested in any suitable manner that is known or developed in the future. In various embodiments, the silver compound including the silver particles 100 is ingested orally, by injection, and/or inhalation (e.g., nebulization, fogging, etc.), among other techniques that are possible, each of which is contemplated herein.

In various embodiments, providing and facilitating daily ingestion of the silver compound including the silver particles 100 is performed to kill the bacteria Clostridium perfringens in the digestive system (e.g., intestines) of the animal. In additional or alternative embodiments, providing and facilitating daily ingestion of the silver compound including the silver particles 100 is performed to prevent and/or treat necrotic enteritis in the animal.

Facilitating daily ingestion of the silver compound including the silver particles 100, in various embodiments, may include facilitating daily ingestion of any suitable amount of the silver compound in the animal's drinking water. In some embodiments, the drinking water includes an amount of the silver compound in the range of about 0.15 ounces per gallon (0.15 oz/gal.) to about sixteen ounces per gallon (16 oz/gal.) of the silver compound, among other amounts and/or ranges of amounts that are less than 0.15 oz/gal. or greater than 16 oz/gal. that are possible, each of which is contemplated herein. In certain embodiments, the drinking water includes about six ounces per gallon (6 oz/gal.) of the silver compound, among other amounts that are less than 6 oz/gal. or greater than 6 oz/gal. that are possible, each of which is contemplated herein.

In various embodiments, facilitating daily ingestion or causing daily ingestion of the silver compound including the silver particles 100 (e.g., via manual administration) may include ingestion of the silver compound including the silver particles 100 any suitable number of times a day. For example, ingesting an amount of the silver compound including the silver particles 100 can include ingestion once a day, two times a day, three times a day, etc., among other quantities that are possible, each of which is contemplated herein. In certain embodiments, facilitating daily ingestion or causing daily ingestion of the silver compound including the silver particles 100 may include the silver compound including the silver particles 100 being added to each filling/refilling of the animal's drinking water and/or each time the drinking water is changed out.

In at least some embodiments in which the silver compound including the silver particles 100 is ingested or caused to be ingested at least twice daily, each ingestion is separated by a predetermined minimum amount of time, which can be any suitable amount of time that can facilitate protecting and/or treating the digestive system of an animal. Example predetermined minimum amounts of time can include, but are not limited to, one hour, two hours, four hours, six hours, eight hours, twelve hours, etc., among other minimum amounts of time that are possible, each of which is contemplated herein.

Referring to FIG. 4, FIG. 4 is a block diagram of another embodiment of a method 400 for using a silver compound to protect the digestive system of an animal (e.g., fowl, livestock, or other animal). At least in the illustrated embodiment, the method 400 can begin by a providing a silver compound to the animal (block 402).

The silver compound can be provided to the animal in any suitable manner that is known or developed in the future. Examples of providing the silver compound to the animal can include, but is not limited to, automatically and/or passively (e.g., via drinking water) and/or manual administration (e.g., by a caregiver), etc., among other techniques/methods that are possible, each of which is contemplated herein.

The silver compound can include any embodiment of the silver particles 100 discussed herein. That is, the silver particles 100 may include nano-silver particles 100, colloidal silver particles 100, ionic silver particles 100, solution silver particles 100, and/or suspension silver particles 100.

In some embodiments, the silver compound includes an amount of silver particles 100 (e.g., Ag particles), which can include any amount of silver particles 100 in the range of about 0.5 ppm to about 100 ppm, by weight and/or volume, as discussed elsewhere herein. In certain embodiments, the amount of silver particles 100 is 10 ppm or 32 ppm, by weight and/or volume, among other amounts that are possible, each of which is contemplated herein.

The method 400 further includes facilitating daily ingestion of the silver compound including the silver particles 100 by the animal as a preventative measure to kill Clostridium perfringens in the animal and/or prevent necrotic enteritis in the animal (block 404). Facilitating daily ingestion of the compound including the silver particles 100 may include any suitable technique and/or method that encourages and/or causes an animal to ingest the silver compound including the silver particles 100 on a daily and/or other similar regular basis. Examples of providing the silver compound to the animal can include, but is not limited to, automatically and/or passively (e.g., via drinking water) and/or manual administration (e.g., by a caregiver), etc., among other techniques/methods that are possible, each of which is contemplated herein.

The silver compound including the silver particles 100 may be ingested in any suitable manner that is known or developed in the future. In various embodiments, the silver compound including the silver particles 100 is ingested orally, by injection, and/or by inhalation, among other techniques that are possible, each of which is contemplated herein.

In various embodiments, providing and facilitating daily ingestion of the silver compound including the silver particles 100 is performed to kill the bacteria Clostridium perfringens in the digestive system (e.g., intestines) of the animal. In additional or alternative embodiments, providing and facilitating daily ingestion of the silver compound including the silver particles 100 is performed to prevent an animal from developing necrotic enteritis.

Facilitating daily ingestion of the silver compound including the silver particles 100, in various embodiments, may include facilitating daily ingestion of any suitable amount of the silver compound in the animal's drinking water. In some embodiments, the drinking water includes an amount of the silver compound in the range of about 0.15 ounces per gallon (0.15 oz/gal.) to about sixteen ounces per gallon (16 oz/gal.) of the silver compound, among other amounts and/or ranges of amounts that are less than 0.15 oz/gal. or greater than 16 oz/gal. that are possible, each of which is contemplated herein. In certain embodiments, the drinking water includes about six ounces per gallon (6 oz/gal.) of the silver compound, among other amounts that are less than 6 oz/gal. or greater than 6 oz/gal. that are possible, each of which is contemplated herein.

In various embodiments, facilitating daily ingestion or causing daily ingestion of the silver compound including the silver particles 100 (e.g., via manual administration) may include ingestion of the silver compound including the silver particles 100 any suitable number of times a day. For example, ingesting an amount of the silver compound including the silver particles 100 can include ingestion once a day, two times a day, three times a day, etc., among other quantities that are possible, each of which is contemplated herein.

In at least some embodiments in which the silver compound including the silver particles 100 is ingested or caused to be ingested at least twice daily, each ingestion is separated by a predetermined minimum amount of time, which can be any suitable amount of time that can facilitate protecting and/or treating the digestive system of the animal. Example predetermined minimum amounts of time can include, but are not limited to, one hour, two hours, four hours, six hours, eight hours, twelve hours, etc., among other minimum amounts of time that are possible, each of which is contemplated herein.

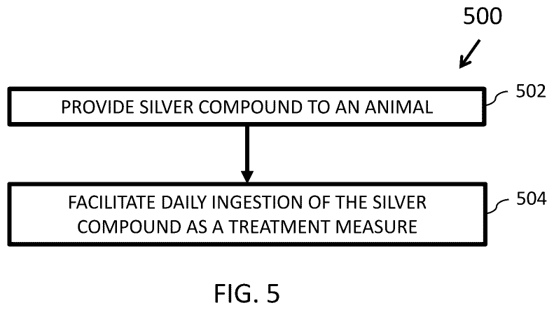

With reference to FIG. 5, FIG. 5 is a block diagram of another embodiment of a method 500 for using a silver compound to protect the digestive system of an animal (e.g., fowl, livestock, or other animal). At least in the illustrated embodiment, the method 500 can begin by a providing a silver compound to an animal (block 502).

The silver compound can be provided to the animal in any suitable manner that is known or developed in the future. Examples of providing the silver compound to the animal can include, but is not limited to, automatically and/or passively (e.g., via drinking water) and/or manual administration (e.g., by a caregiver), etc., among other techniques/methods that are possible, each of which is contemplated herein.

The silver compound can include any embodiment of the silver particles 100 discussed herein. That is, the silver particles 100 may include nano-silver particles 100, colloidal silver particles 100, ionic silver particles 100, solution silver particles 100, and/or suspension silver particles 100.

In some embodiments, the silver compound includes an amount of silver particles 100 (e.g., Ag particles), which can include any amount of silver particles 100 in the range of about 0.5 ppm to about 100 ppm, by weight and/or volume, as discussed elsewhere herein. In certain embodiments, the amount of silver particles 100 is 10 ppm or 32 ppm, by weight and/or volume, among other amounts that are possible, each of which is contemplated herein.

The method 500 further includes facilitating daily ingestion of the silver compound including the silver particles 100 by the animal as a treatment measure to kill Clostridium perfringens in the animal and/or treat necrotic enteritis in the animal (block 504). Facilitating daily ingestion of the compound including the silver particles 100 may include any suitable technique and/or method that encourages and/or causes an animal to ingest the silver compound including the silver particles 100 on a daily and/or other similar regular basis. Examples of providing the silver compound to the animal can include, but is not limited to, automatically and/or passively (e.g., via drinking water) and/or manual administration (e.g., by a caregiver), etc., among other techniques/methods that are possible, each of which is contemplated herein.

The silver compound including the silver particles 100 may be ingested in any suitable manner that is known or developed in the future. In various embodiments, the silver compound including the silver particles 100 is ingested orally, by injection, and/or by inhalation, among other techniques that are possible, each of which is contemplated herein.

In various embodiments, providing and facilitating daily ingestion of the silver compound including the silver particles 100 is performed to kill the bacteria Clostridium perfringens in the digestive system (e.g., intestines) of the animal. In additional or alternative embodiments, providing and facilitating daily ingestion of the silver compound including the silver particles 100 is performed to treat an animal suffering from necrotic enteritis.

Facilitating daily ingestion of the silver compound including the silver particles 100, in various embodiments, may include facilitating daily ingestion of any suitable amount of the silver compound in the animal's drinking water. In some embodiments, the drinking water includes an amount of the silver compound in the range of about 0.15 ounces per gallon (0.15 oz/gal.) to about sixteen ounces per gallon (16 oz/gal.) of the silver compound, among other amounts and/or ranges of amounts that are less than 0.15 oz/gal. or greater than 16 oz/gal. that are possible, each of which is contemplated herein. In certain embodiments, the drinking water includes about six ounces per gallon (6 oz/gal.) of the silver compound, among other amounts that are less than 6 oz/gal. or greater than 6 oz/gal. that are possible, each of which is contemplated herein.

In various embodiments, facilitating daily ingestion or causing daily ingestion of the silver compound including the silver particles 100 (e.g., via manual administration) may include ingestion of the silver compound including the silver particles 100 any suitable number of times a day. For example, ingesting an amount of the silver compound including the silver particles 100 can include ingestion once a day, two times a day, three times a day, etc., among other quantities that are possible, each of which is contemplated herein.

In at least some embodiments in which the silver compound including the silver particles 100 is ingested or caused to be ingested at least twice daily, each ingestion is separated by a predetermined minimum amount of time, which can be any suitable amount of time that can facilitate protecting and/or treating the digestive system of the animal. Example predetermined minimum amounts of time can include, but are not limited to, one hour, two hours, four hours, six hours, eight hours, twelve hours, etc., among other minimum amounts of time that are possible, each of which is contemplated herein.

While the various embodiments of methods 300, 400, and 500 in FIGS. 3, 4, and 5, respectively, are disclosed with reference to an animal (e.g., fowl, livestock, or other animal), the various embodiments of methods 300, 400, and 500 are not limited to the example types of animals. That is, the various embodiments of methods 300, 400, and/or 500 can be specifically applied to chickens, ducks, turkeys, geese, pigs, cattle, sheep, goats, horses, etc., among other fowl and/or livestock that are possible, each of which is contemplated herein. In addition, various other embodiments of methods 300, 400, and/or 500 include protecting the digestive system of any other animal and/or type of animal.

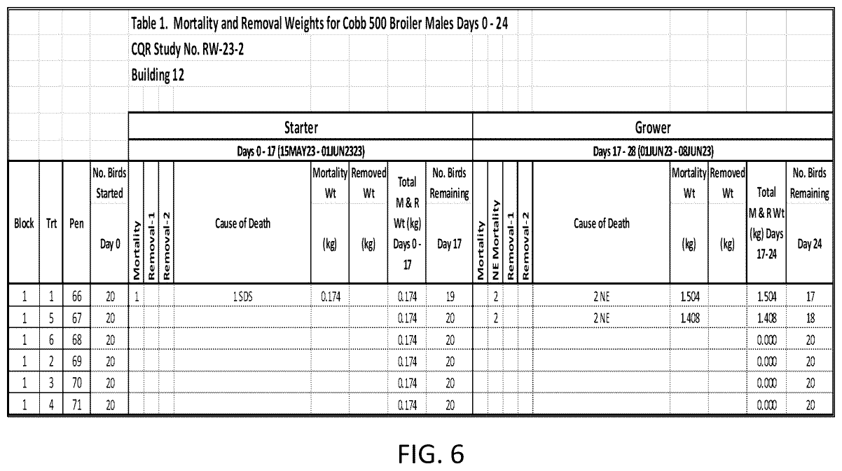

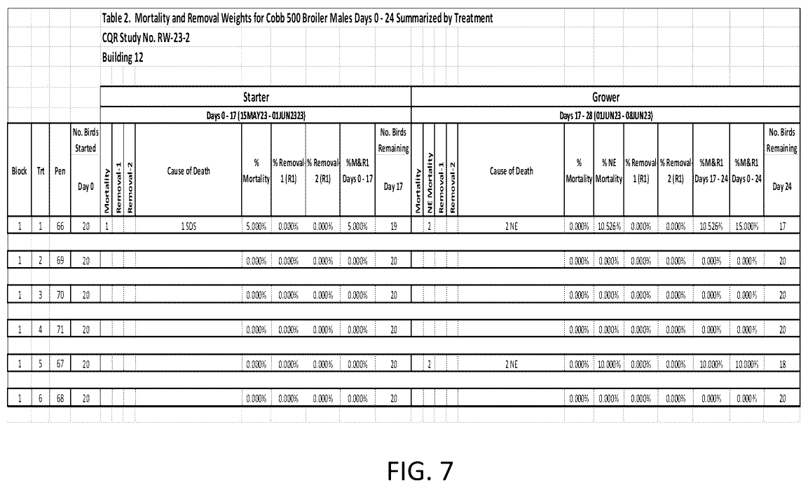

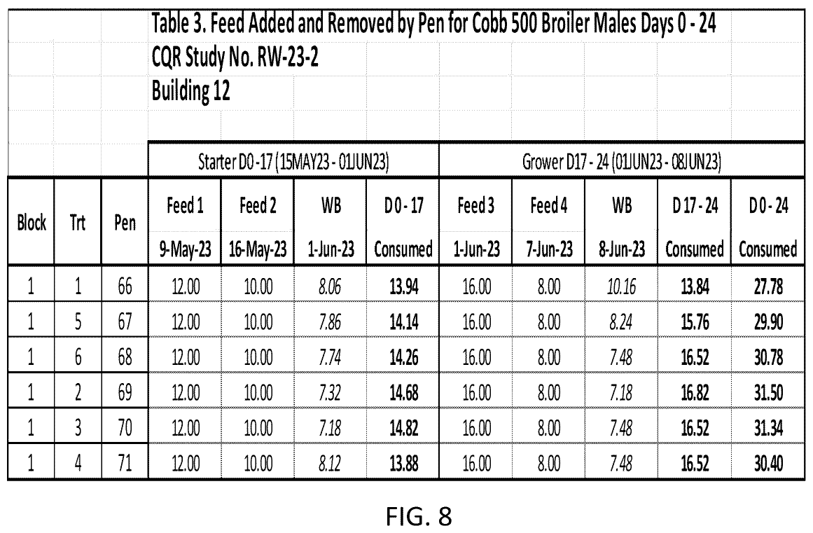

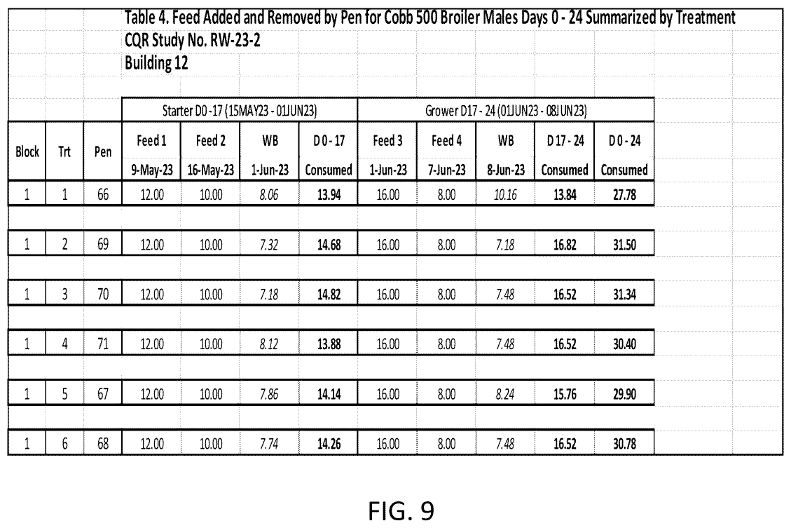

FIG. 6 is a table illustrating mortality and removal weights for chickens (e.g., Cobb 500 Broiler males) 0-24 days old. FIG. 7 is a table illustrating mortality and removal weights for chickens (e.g., Cobb 500 Broiler males) 0-24 days old summarized by treatment. FIG. 8 is a table illustrating feed added and removed for chickens (e.g., Cobb 500 Broiler males) 0-24 days old. FIG. 9 is a table illustrating feed added and removed for chickens (e.g., Cobb 500 Broiler males) 0-24 days old summarized by treatment.

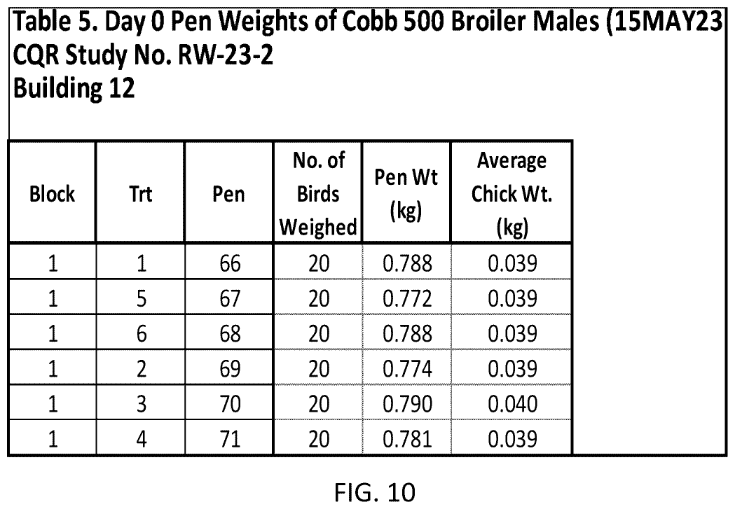

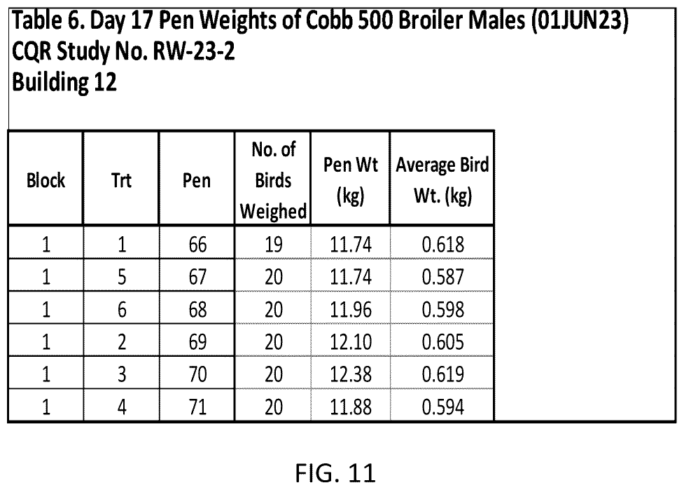

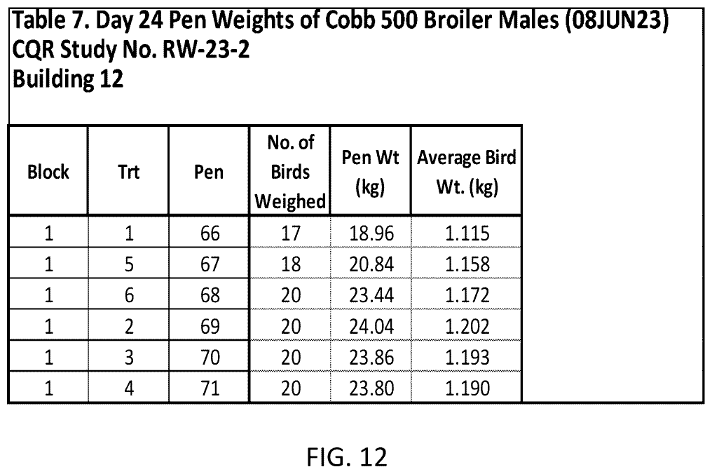

FIG. 10 is a table illustrating day 0 pen weights of chickens (e.g., Cobb 500 Broiler males). FIG. 11 is a table illustrating day 17 pen weights of chickens (e.g., Cobb 500 Broiler males). FIG. 12 is a table illustrating day 24 pen weights of chickens (e.g., Cobb 500 Broiler males).

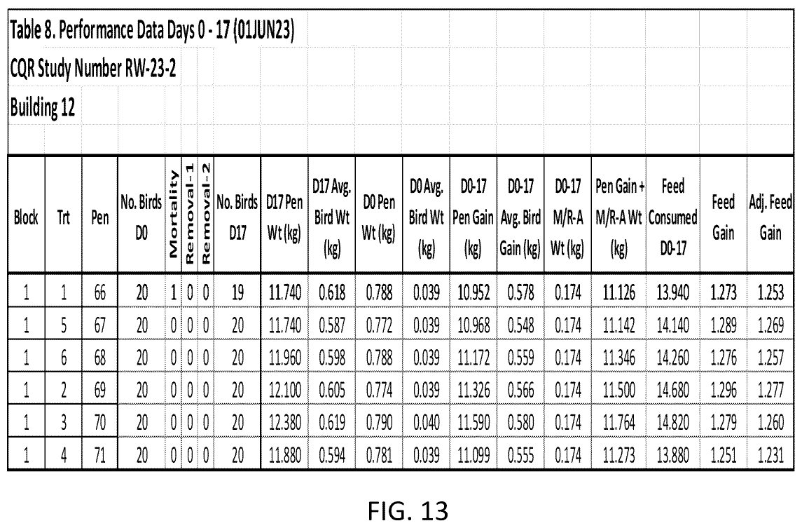

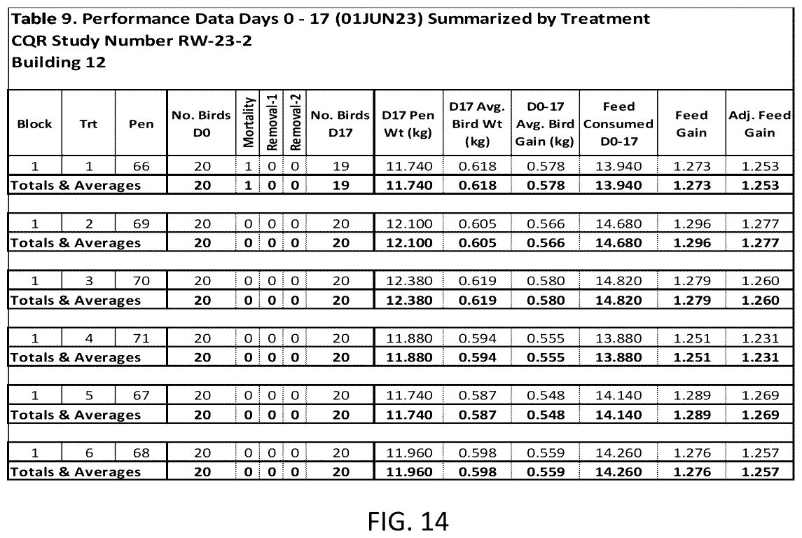

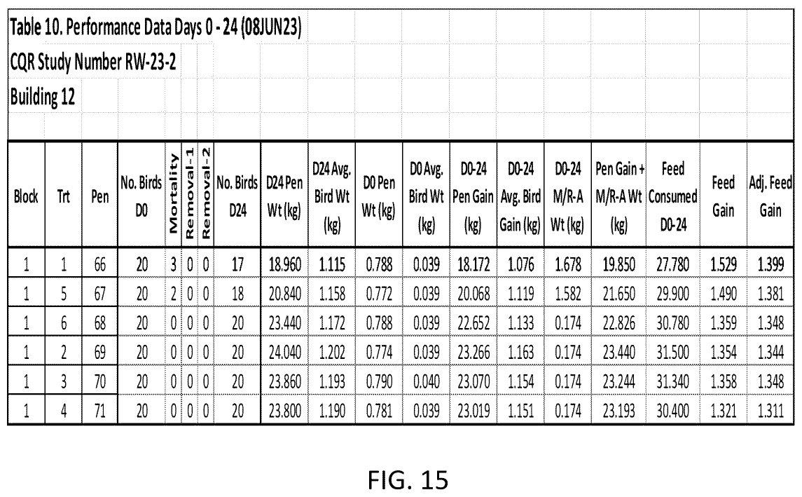

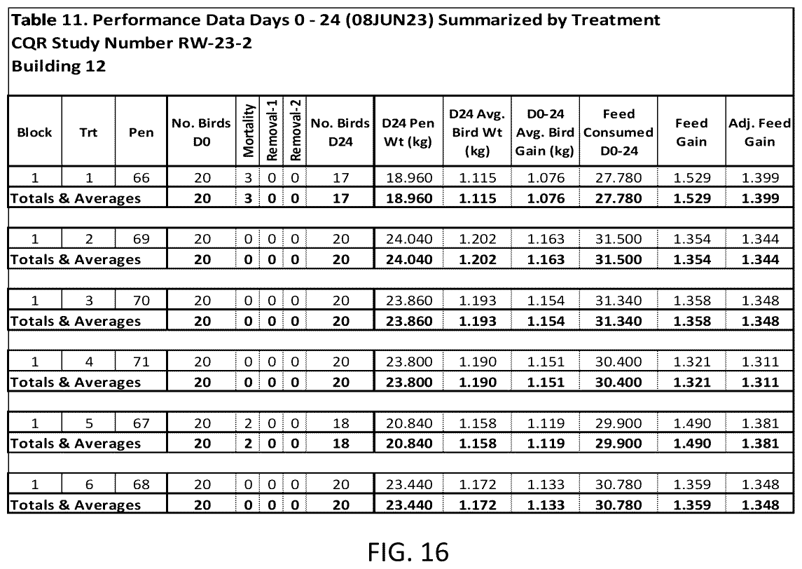

FIG. 13 is a table illustrating performance data of chickens (e.g., Cobb 500 Broiler males) for days 0-17. FIG. 14 is a table illustrating performance data of chickens (e.g., Cobb 500 Broiler males) for days 0-17 summarized by treatment. FIG. 15 is a table illustrating performance data of chickens (e.g., Cobb 500 Broiler males) for days 0-24. FIG. 16 is a table illustrating performance data of chickens (e.g., Cobb 500 Broiler males) for days 0-24 summarized by treatment.

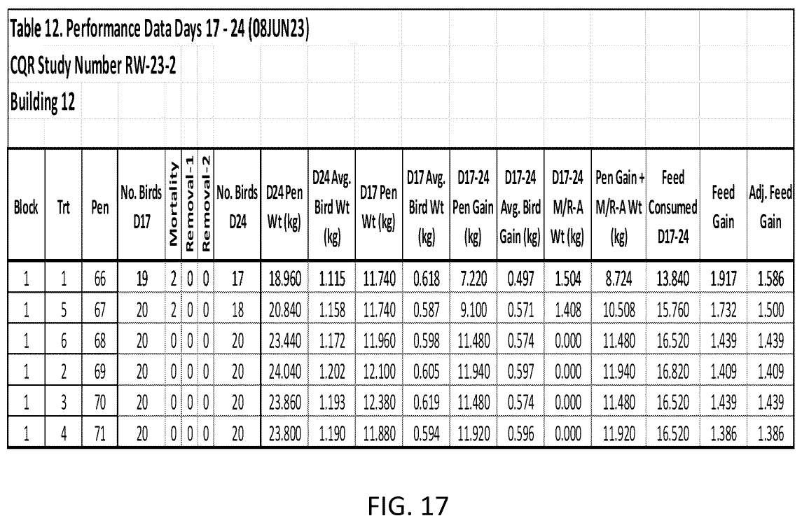

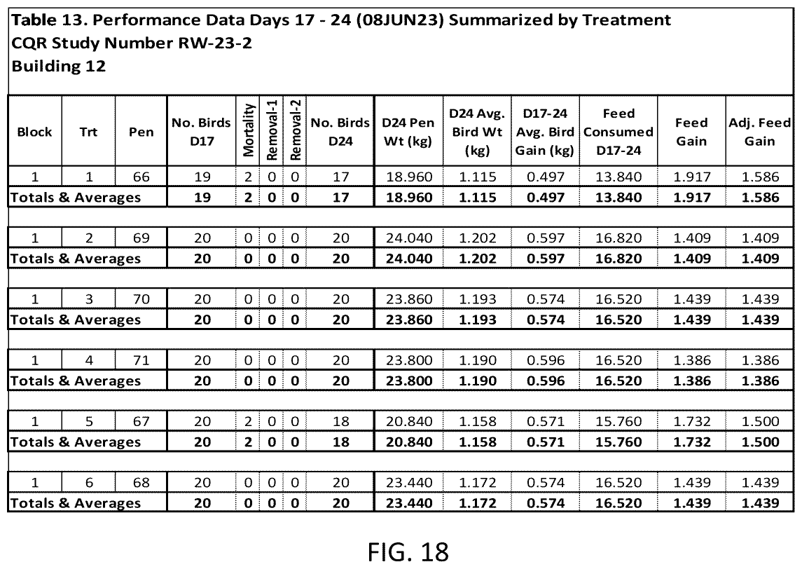

FIG. 17 is a table illustrating performance data of chickens (e.g., Cobb 500 Broiler males) for days 17-24. FIG. 18 is a table illustrating performance data of chickens (e.g., Cobb 500 Broiler males) for days 17-24 summarized by treatment.

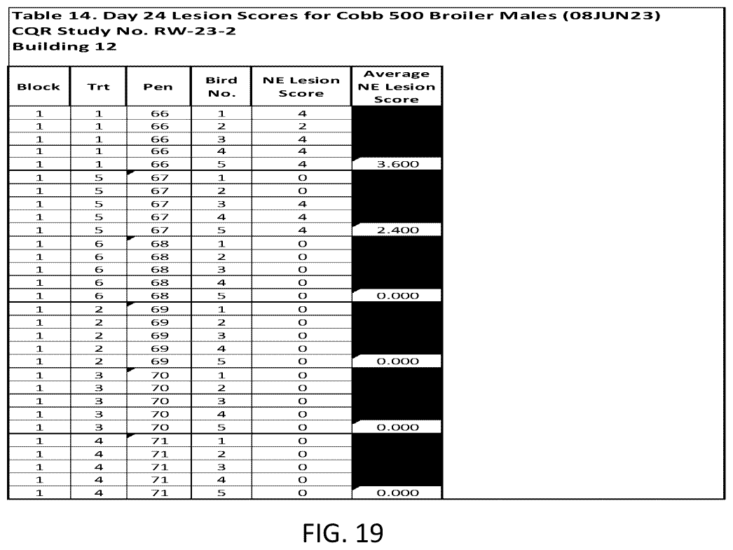

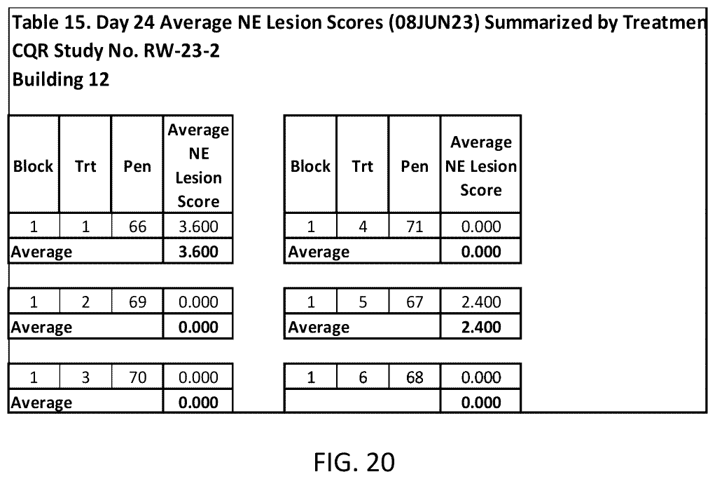

FIG. 19 is a table illustrating day 24 average necrotic enteritis lesion scores for chickens (e.g., Cobb 500 Broiler males). FIG. 20 is a table illustrating day 24 average necrotic enteritis lesion scores for chickens (e.g., Cobb 500 Broiler males) summarized by treatment.

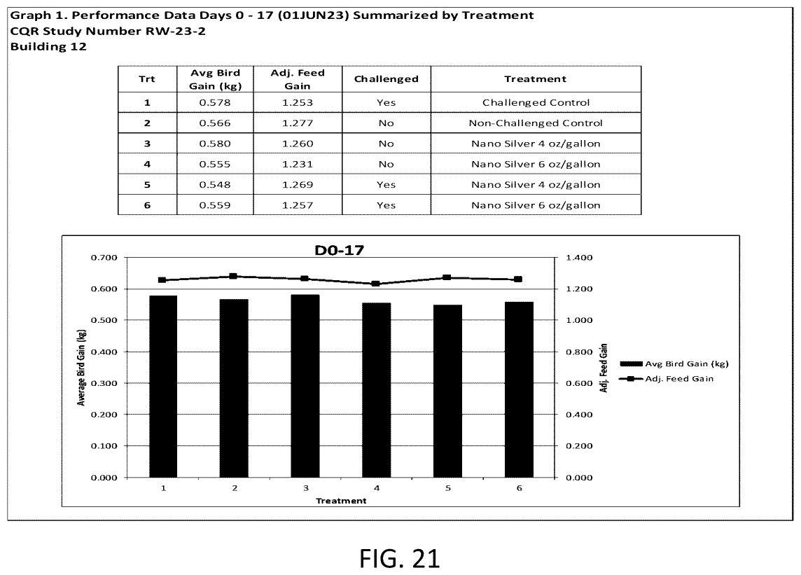

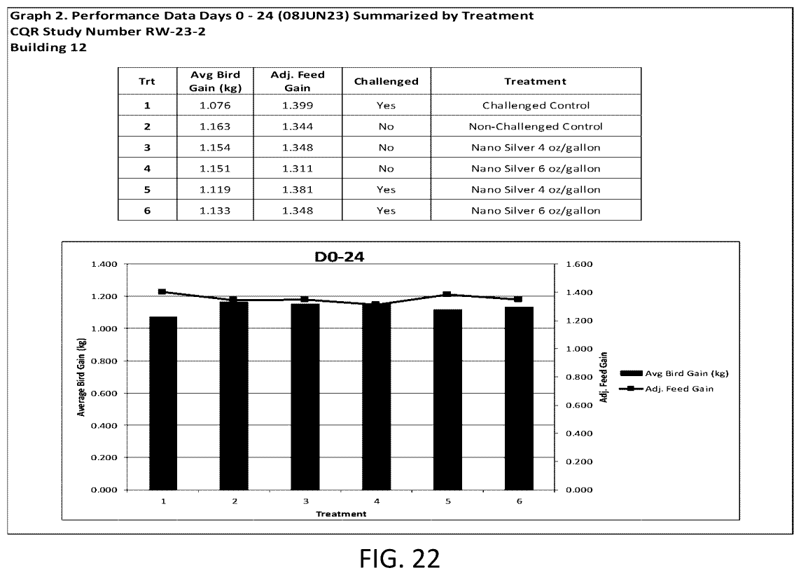

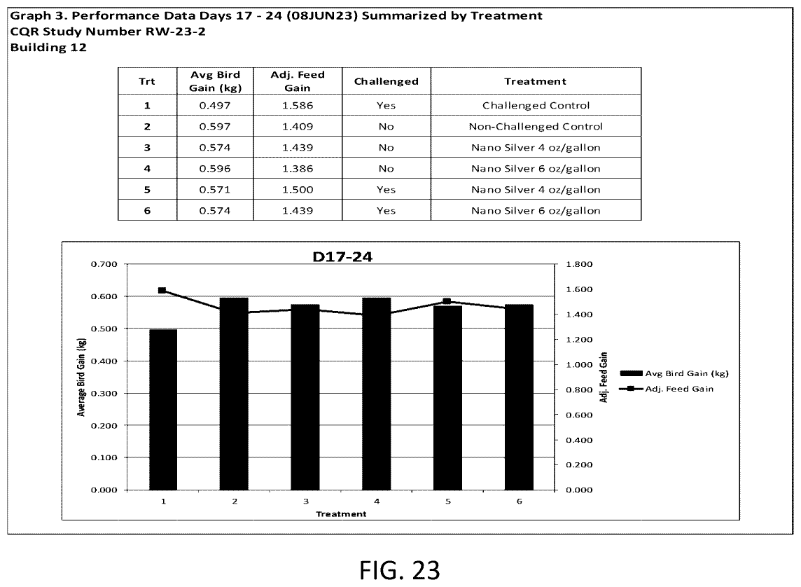

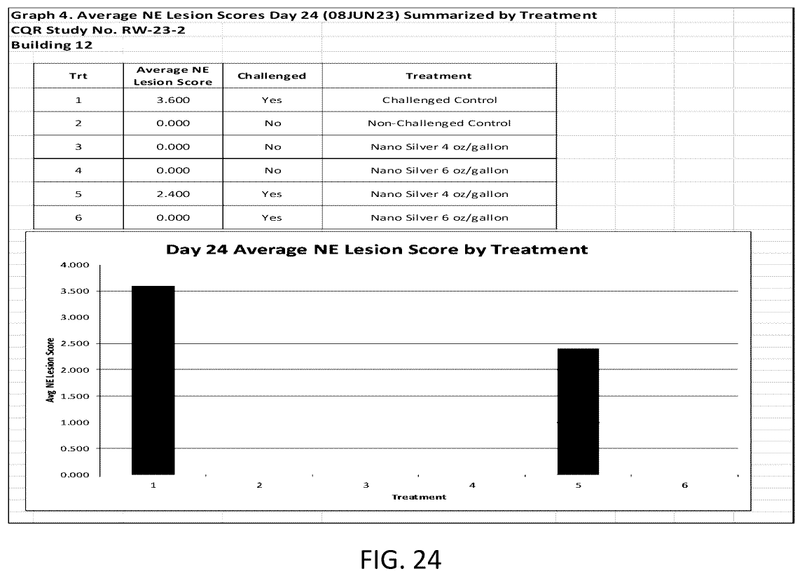

FIG. 21 is a graph illustrating performance data of chickens (e.g., Cobb 500 Broiler males) for days 0-17 summarized by treatment. FIG. 22 is a graph illustrating performance data of chickens (e.g., Cobb 500 Broiler males) for days 0-24 summarized by treatment. FIG. 23 is a graph illustrating performance data of chickens (e.g., Cobb 500 Broiler males) for days 17-24 summarized by treatment. FIG. 24 is a graph illustrating average necrotic enteritis lesion scores of chickens (e.g., Cobb 500 Broiler males) summarized by treatment.

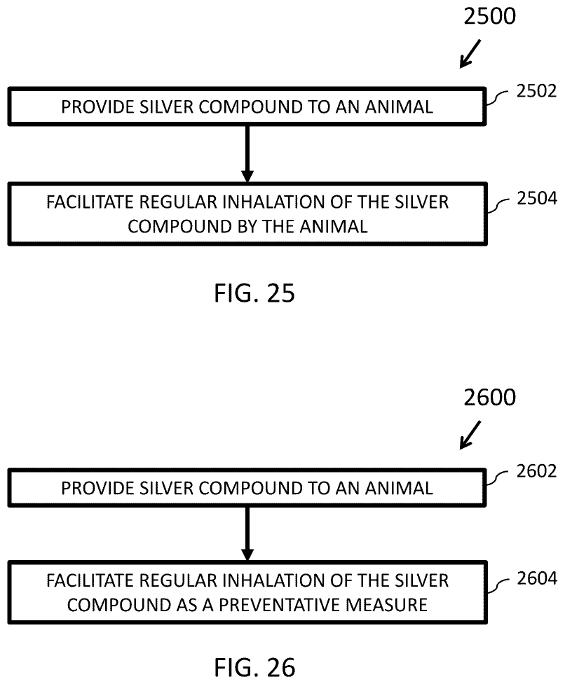

With reference to FIG. 25, FIG. 25 is a block diagram of one embodiment of a method 2500 for using a silver compound to protect the respiratory system of an animal (e.g., fowl, livestock, or other animal) from a pathogenic bacterium, prevent at least one type of respiratory disease, and/or treat at least one type of respiratory disease in the animal. At least in the illustrated embodiment, the method 2500 can begin by a providing a silver compound to the animal (block 2502).

The silver compound can be provided to the animal in any suitable manner that is known or developed in the future. Examples of providing the silver compound to the animal can include, but is not limited to, automatically and/or passively (e.g., via inhaled air and/or breathable air) and/or manual administration by a caregiver (e.g., nebulization, fogging, misting, spraying, etc.), among other techniques/methods that are possible, each of which is contemplated herein.

The silver compound can include any embodiment of the silver particles 100 discussed herein. That is, the silver particles 100 may include nano-silver particles 100, colloidal silver particles 100, ionic silver particles 100, solution silver particles 100, and/or suspension silver particles 100.

In some embodiments, the silver compound includes an amount of silver particles 100 (e.g., Ag particles), which can include any amount of silver particles 100 in the range of about 0.5 ppm to about 100 ppm, by weight and/or volume, as discussed elsewhere herein. In certain embodiments, the amount of silver particles 100 is 10 ppm or 32 ppm, by weight and/or volume, among other amounts that are possible, each of which is contemplated herein.

The method 2500 further includes facilitating regular inhalation (e.g., daily, weekly, bi-weekly, monthly, etc.) of the silver compound including the silver particles 100 by the animal (block 2504). Facilitating regular inhalation of the compound including the silver particles 100 may include any suitable technique and/or method that encourages and/or causes an animal to inhale the silver compound including the silver particles 100 on a daily, weekly, biweekly, monthly, etc. and/or other similar regular basis. Examples of providing the silver compound to the animal can include, but is not limited to, automatically and/or passively (e.g., via inhaled/breathable air) and/or manual administration by a caregiver (e.g., nebulization, fogging, misting, spraying, etc.), among other techniques/methods that are possible, each of which is contemplated herein.

The silver compound including the silver particles 100 may be inhaled in any suitable manner that is known or developed in the future. In various embodiments, the silver compound including the silver particles 100 is inhaled via nebulization, fogging, misting, spraying, etc., among other techniques that are possible, each of which is contemplated herein.

In various embodiments, providing and facilitating regular inhalation of the silver compound including the silver particles 100 is performed to kill one or more types of pathogenic bacteria in the respiratory system of the animal. In additional or alternative embodiments, providing and facilitating regular inhalation of the silver compound including the silver particles 100 is performed to prevent and/or treat at least one respiratory disease and/or one or more types of respiratory diseases in the animal.

Facilitating regular inhalation of the silver compound including the silver particles 100, in various embodiments, may include facilitating regular inhalation of any suitable amount of the silver compound in the animal breathable air (e.g., via fogging, spraying, nebulizing, misting, etc.). In various embodiments, the silver compound including the silver particles 100 in the fog, mist, spray, and/or droplets, etc., in the animal's breathable air can be diluted or undiluted.

In various embodiments, facilitating regular inhalation or causing regular inhalation of the silver compound including the silver particles 100 (e.g., via manual administration and/or in the breathable air) may include inhalation of the silver compound including the silver particles 100 any suitable number of times a day. For example, inhaling an amount of the silver compound including the silver particles 100 can include inhalation once a day, two times a day, three times a day, etc., among other quantities that are possible, each of which is contemplated herein. In certain embodiments, facilitating regular inhalation or causing daily inhalation of the silver compound including the silver particles 100 may include the silver compound including the silver particles 100 being added to the animal's breathable air and/or living environment and/or manually providing the fog, mist, spray, and/or droplets, etc. in the silver compound including the silver particles 100 to the animal.

In at least some embodiments, each inhalation is separated by a predetermined minimum amount of time, which can be any suitable amount of time that can facilitate protecting and/or treating the respiratory system of an animal. Example predetermined minimum amounts of time can include, but are not limited to, one hour, two hours, four hours, six hours, eight hours, twelve hours, a day, a week, two weeks, a month etc., among other minimum amounts of time that are possible, each of which is contemplated herein.

Referring to FIG. 26, FIG. 26 is a block diagram of another embodiment of a method 2600 for using a silver compound to protect the respiratory system of an animal (e.g., a livestock animal or other animal). At least in the illustrated embodiment, the method 2600 can begin by a providing a silver compound to the animal (block 2602).

The silver compound can be provided to the animal in any suitable manner that is known or developed in the future. Examples of providing the silver compound to the animal can include, but is not limited to, automatically and/or passively (e.g., via inhaled and/or breathable air) and/or manual administration (e.g., by a caregiver), etc., among other techniques/methods that are possible, each of which is contemplated herein.

The silver compound can include any embodiment of the silver particles 100 discussed herein. That is, the silver particles 100 may include nano-silver particles 100, colloidal silver particles 100, ionic silver particles 100, solution silver particles 100, and/or suspension silver particles 100.

In some embodiments, the silver compound includes an amount of silver particles 100 (e.g., Ag particles), which can include any amount of silver particles 100 in the range of about 0.5 ppm to about 100 ppm, by weight and/or volume, as discussed elsewhere herein. In certain embodiments, the amount of silver particles 100 is 10 ppm or 32 ppm, by weight and/or volume, among other amounts that are possible, each of which is contemplated herein.

The method 2600 further includes facilitating regular inhalation of the silver compound including the silver particles 100 by the animal as a preventative measure to kill one or more pathogenic bacterium and/or one or more types of pathogenic bacteria in the respiratory system of the animal and/or prevent one or more respiratory diseases and/or one or more types of respiratory disease in the animal (block 2604). Facilitating regular inhalation of the compound including the silver particles 100 may include any suitable technique and/or method that encourages and/or causes an animal to inhale the silver compound including the silver particles 100 on a regular basis. Examples of providing the silver compound to the animal can include, but is not limited to, automatically and/or passively (e.g., via inhaled air and/or breathable air) and/or manual administration (e.g., by a caregiver), etc., among other techniques/methods that are possible, each of which is contemplated herein.

The silver compound including the silver particles 100 may be inhaled in any suitable manner that is known or developed in the future. In various embodiments, the silver compound including the silver particles 100 is inhaled by inhalation of a fog, a mist, a spray, droplets, etc. that includes the silver compound including the silver particles 100, among other techniques that are possible, each of which is contemplated herein.

In various embodiments, providing and facilitating regular inhalation of the silver compound including the silver particles 100 is performed to kill one or more pathogenic bacteria and/or one or more types of pathogenic bacteria in the respiratory system of the animal. In additional or alternative embodiments, providing and facilitating regular inhalation of the silver compound including the silver particles 100 is performed to prevent an animal from developing one or more respiratory diseases and/or one or more types of respiratory disease.

Facilitating regular inhalation of the silver compound including the silver particles 100, in various embodiments, may include facilitating regular inhalation of any suitable amount of the silver compound in the animal breathable air (e.g., via fogging, spraying, nebulizing, misting, etc.). In various embodiments, the silver compound including the silver particles 100 in the fog, mist, spray, and/or droplets, etc., in the animal's breathable air can be diluted or undiluted.

In various embodiments, facilitating regular inhalation or causing regular inhalation of the silver compound including the silver particles 100 (e.g., via manual administration and/or in the breathable air) may include inhalation of the silver compound including the silver particles 100 any suitable number of times. For example, inhaling an amount of the silver compound including the silver particles 100 can include regular inhalation occurring once a day, two times a day, three times a day, etc., among other quantities that are possible, each of which is contemplated herein. In certain embodiments, facilitating regular inhalation or causing regular inhalation of the silver compound including the silver particles 100 may include the silver compound including the silver particles 100 being added to the animal's breathable air and/or living environment and/or manually providing the fog, mist, spray, and/or droplets, etc. in the silver compound including the silver particles 100 to the animal.

In at least some embodiments, each inhalation is separated by a predetermined minimum amount of time, which can be any suitable amount of time that can facilitate protecting the respiratory system of an animal and/or preventing the animal from developing a respiratory disease. Example predetermined minimum amounts of time can include, but are not limited to, one hour, two hours, four hours, six hours, eight hours, twelve hours, a day, a week, two weeks, a month, etc., among other minimum amounts of time that are possible, each of which is contemplated herein.

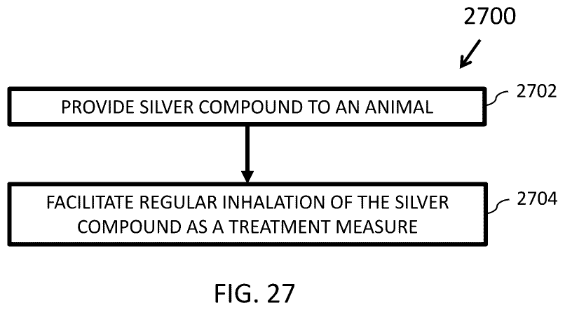

With reference to FIG. 27, FIG. 27 is a block diagram of another embodiment of a method 2700 for using a silver compound to protect the respiratory system of an animal (e.g., a livestock animal or other animal). At least in the illustrated embodiment, the method 2700 can begin by a providing a silver compound to the animal (block 2702).

The silver compound can be provided to the animal in any suitable manner that is known or developed in the future. Examples of providing the silver compound to the animal can include, but is not limited to, automatically and/or passively (e.g., via inhaled and/or breathable air) and/or manual administration (e.g., by a caregiver), etc., among other techniques/methods that are possible, each of which is contemplated herein.

The silver compound can include any embodiment of the silver particles 100 discussed herein. That is, the silver particles 100 may include nano-silver particles 100, colloidal silver particles 100, ionic silver particles 100, solution silver particles 100, and/or suspension silver particles 100.

In some embodiments, the silver compound includes an amount of silver particles 100 (e.g., Ag particles), which can include any amount of silver particles 100 in the range of about 0.5 ppm to about 100 ppm, by weight and/or volume, as discussed elsewhere herein. In certain embodiments, the amount of silver particles 100 is 10 ppm or 32 ppm, by weight and/or volume, among other amounts that are possible, each of which is contemplated herein.

The method 2600 further includes facilitating regular inhalation of the silver compound including the silver particles 100 by the animal as a treatment measure to kill one or more pathogenic bacterium and/or one or more types of pathogenic bacteria in the respiratory system of the animal and/or treat one or more respiratory diseases and/or one or more types of respiratory disease in the animal (block 2704). Facilitating regular inhalation of the compound including the silver particles 100 may include any suitable technique and/or method that encourages and/or causes an animal to inhale the silver compound including the silver particles 100 on a regular basis. Examples of providing the silver compound to the animal can include, but is not limited to, automatically and/or passively (e.g., via inhaled air and/or breathable air) and/or manual administration (e.g., by a caregiver), etc., among other techniques/methods that are possible, each of which is contemplated herein.

The silver compound including the silver particles 100 may be inhaled in any suitable manner that is known or developed in the future. In various embodiments, the silver compound including the silver particles 100 is inhaled by inhalation of a fog, a mist, a spray, droplets, etc. that includes the silver compound including the silver particles 100, among other techniques that are possible, each of which is contemplated herein.

In various embodiments, providing and facilitating regular inhalation of the silver compound including the silver particles 100 is performed to kill one or more pathogenic bacteria and/or one or more types of pathogenic bacteria in the respiratory system of the animal. In additional or alternative embodiments, providing and facilitating regular inhalation of the silver compound including the silver particles 100 is performed to treat an animal afflicted with one or more respiratory diseases and/or one or more types of respiratory disease.

Facilitating regular inhalation of the silver compound including the silver particles 100, in various embodiments, may include facilitating regular inhalation of any suitable amount of the silver compound in the animal breathable air (e.g., via fogging, spraying, nebulizing, misting, etc.). In various embodiments, the silver compound including the silver particles 100 in the fog, mist, spray, and/or droplets, etc., in the animal's breathable air can be diluted or undiluted.

In various embodiments, facilitating regular inhalation or causing regular inhalation of the silver compound including the silver particles 100 (e.g., via manual administration and/or in the breathable air) may include inhalation of the silver compound including the silver particles 100 any suitable number of times a day. For example, inhaling an amount of the silver compound including the silver particles 100 can include inhalation once a day, two times a day, three times a day, etc., among other quantities that are possible, each of which is contemplated herein. In certain embodiments, facilitating regular inhalation or causing regular inhalation of the silver compound including the silver particles 100 may include the silver compound including the silver particles 100 being added to the animal's breathable air and/or living environment and/or manually providing the fog, mist, spray, and/or droplets, etc. in the silver compound including the silver particles 100 to the animal.

In at least some embodiments, each inhalation is separated by a predetermined minimum amount of time, which can be any suitable amount of time that can facilitate protecting the respiratory system of an animal and/or treating a respiratory disease in the animal. Example predetermined minimum amounts of time can include, but are not limited to, one hour, two hours, four hours, six hours, eight hours, twelve hours, a day, a week, two weeks, a month, etc., among other minimum amounts of time that are possible, each of which is contemplated herein.

While the various embodiments of methods 2500, 2600, and 2700 in FIGS. 25, 26, and 27, respectively, are disclosed with reference to an animal (e.g., fowl, livestock, or other animal), the various embodiments of methods 2500, 2600, and 2700 are not limited to the example types of animals. That is, the various embodiments of methods 2500, 2600, and/or 2700 can be specifically applied to chickens, ducks, turkeys, geese, pigs, cattle, sheep, goats, horses, etc., among other fowl and/or livestock that are possible, each of which is contemplated herein. In addition, various other embodiments of methods 2500, 2600, and/or 2700 include protecting the digestive system of any other animal and/or type of animal.

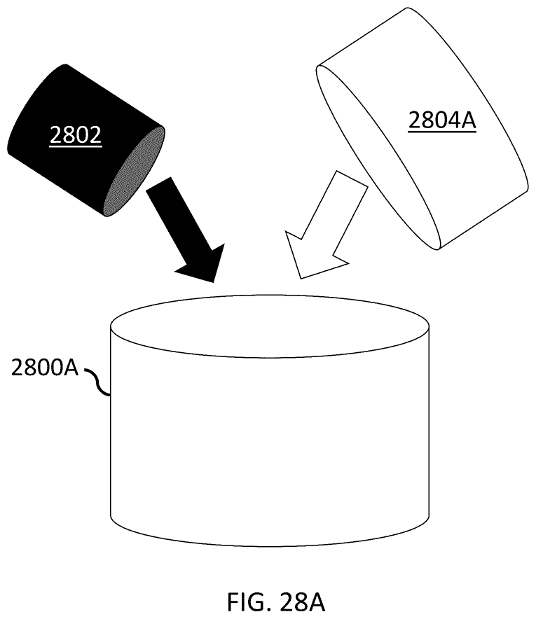

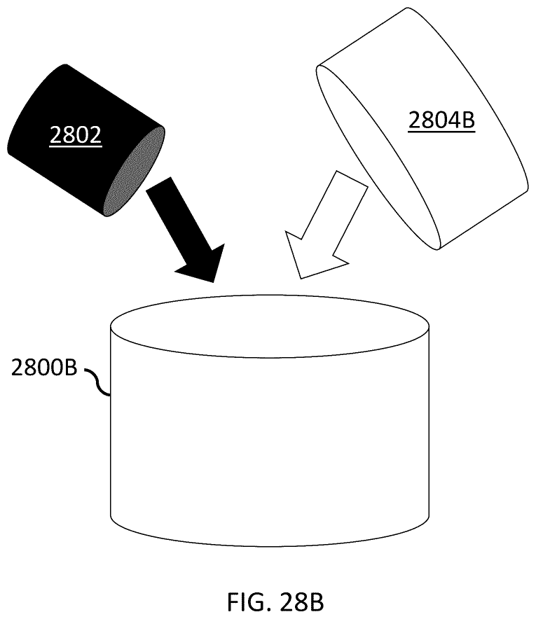

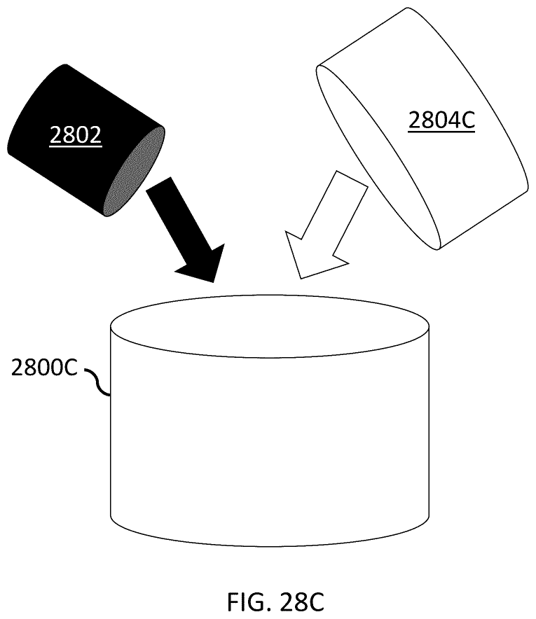

With reference to FIG. 28A, FIG. 28A is a diagram illustrating the creation of a medicated food 2800A for an animal (e.g., fowl, livestock, or other animal). In various embodiments, the medicated food 2800A is created by combining a compound 2802 including silver particles and a feed 2804A.

The compound 2802 including silver particles may include any of the compounds including silver particles discussed above or elsewhere herein. In addition, the silver particles may include any of the embodiments of silver particles discussed above or elsewhere herein. In various embodiments, the silver particles include the silver particles 100 discussed above. Accordingly, the compound 2802 can include nano-silver particles, colloidal silver particles, ionic silver particles, solution silver particles, and/or suspension silver particles.

A feed 2804A may include any food that is known or developed in the future suitable for an animal (e.g., fowl, livestock, or other animal). Examples of a feed 2804A include, but are not limited to, one or more grains (e.g., corn, corn meal, soybeans, soybean meal, wheat, wheat middlings, peas, oats, etc.), hay, alfalfa, fish meal, kelp, aragonite, leafy greens, beans, one or more cereals, fruits, vegetables, insects, insect meal, etc., and combinations thereof, among other food(s) that is/are suitable for an animal (e.g., fowl, livestock, and/or other animal).

In various embodiments, the ratio of compound 2802 including silver particles and feed 2804A is in the range of about 1:50 (1 part compound 2802 to 50 parts feed 2804A) to about 1:10 (1 part compound 2802 to 10 parts feed 2804A), among other ratios and/or ranges that are possible, each of which is contemplated herein. In certain embodiments, the ratio of compound 2802 including silver particles and feed 2804A is about 1:30 (1 part compound 2802 to 30 parts feed 2804), among other ratios that are possible, each of which is contemplated herein.

The medicated food 2800A, in various embodiments, includes an amount of the compound 2802 including silver particles in the range of about 0.1 ounces to about 1 ounce per pound, by weight and/or volume. In certain embodiments, the medicated food 2800 includes about 0.5 ounces of the compound 2802 including silver particles per pound, by weight and/or volume.

The compound 2802 including silver particles may be combined with the feed 2804A using any suitable method and/or technique that is/are known or developed in the future. In some embodiments, the compound 2802 including silver particles is mixed with and/or into the feed 2804A to create the medicated food 2800A. In other embodiments, the compound 2802 including silver particles is sprayed (e.g., topically) onto the feed 2804A to create the medicated food 2800A. In further embodiments, the compound 2802 including silver particles is mixed with/into the feed 2804A and is also sprayed onto the feed 2804A to create the medicated food 2800A.

Referring to FIG. 28B, FIG. 28B is a diagram illustrating the creation of a medicated starter food 2800B for an animal (e.g., fowl, livestock, or other animal). In various embodiments, the medicated starter food 2800B is created by combining a compound 2802 including silver particles and a feed 2804B.

The compound 2802 including silver particles may include any of the compounds including silver particles discussed above or elsewhere herein. In addition, the silver particles may include any of the embodiments of silver particles discussed above or elsewhere herein. In various embodiments, the silver particles include the silver particles 100 discussed above. Accordingly, the compound 2802 can include nano-silver particles, colloidal silver particles, ionic silver particles, solution silver particles, and/or suspension silver particles.

A feed 2804B may include any food that is known or developed in the future suitable for a juvenile animal (e.g., fowl, livestock, or other animal). Examples of a feed 2804B for an animal include, but are not limited to, one or more grains (e.g., corn, corn meal, soybeans, soybean meal, wheat, wheat middlings, peas, oats, etc.), fish meal, kelp, aragonite, leafy greens, beans, one or more cereals, fruits, vegetables, insects, insect meal, etc., and combinations thereof, among other food(s) that is/are suitable for a juvenile animal (e.g., fowl, livestock, and/or other animal).

In various embodiments, the feed 2804B can include any suitable nutrients (e.g., vitamins, minerals, supplements, etc.) specifically designed and/or tailored for a juvenile animal. In additional or alternative embodiments, the feed 2804B can include any suitable texture and/or size (e.g., a smaller size) specifically designed and/or tailored for a juvenile animal.

In various embodiments, the ratio of compound 2802 including silver particles and feed 2804B is in the range of about 1:50 (1 part compound 2802 to 50 parts feed 2804B) to about 1:10 (1 part compound 2802 to 10 parts feed 2804B), among other ratios and/or ranges that are possible, each of which is contemplated herein. In certain embodiments, the ratio of compound 2802 including silver particles and feed 2804B is about 1:30 (1 part compound 2802 to 30 parts feed 2804B), among other ratios that are possible, each of which is contemplated herein.

The medicated starter food 2800B, in various embodiments, includes an amount of the compound 2802 including silver particles in the range of about 0.1 ounces to about 1 ounce per pound, by weight and/or volume. In certain embodiments, the medicated starter food 2800B includes about 0.5 ounces of the compound 2802 including silver particles per pound, by weight and/or volume.

The compound 2802 including silver particles may be combined with the feed 2804B using any suitable method and/or technique that is/are known or developed in the future. In some embodiments, the compound 2802 including silver particles is mixed with and/or into the feed 2804B to create the medicated starter food 2800B. In other embodiments, the compound 2802 including silver particles is sprayed (e.g., topically) onto the feed 2804B to create the medicated starter food 2800B. In further embodiments, the compound 2802 including silver particles is mixed with/into the feed 2804B and is also sprayed onto the feed 2804B to create the medicated starter food 2800B.

The medicated starter food 2800B may be fed to a juvenile animal until the animal reaches adulthood, which can be any amount of time and/or age of the animal. In various embodiments, the medicated starter food 2800B may be fed to, for example, a juvenile chicken for the first 0-21 days of its life, among other number of days that are less than 21 days or greater than 21 days that are possible, each of which is contemplated herein. In some embodiments, the medicated starter food 2800B may be fed to, for example, a juvenile chicken for the first 17 days of its life, among other number of days that are greater than 17 days or less than 17 days that are possible, each of which is contemplated herein.

With reference to FIG. 28C, FIG. 28C is a diagram illustrating the creation of a medicated grower food 2800C for an animal (e.g., fowl, livestock, or other animal). In various embodiments, the medicated grower food 2800C is created by combining a compound 2802 including silver particles and a feed 2804C.

The compound 2802 including silver particles may include any of the compounds including silver particles discussed above or elsewhere herein. In addition, the silver particles may include any of the embodiments of silver particles discussed above or elsewhere herein. In various embodiments, the silver particles include the silver particles 100 discussed above. Accordingly, the compound 2802 can include nano-silver particles, colloidal silver particles, ionic silver particles, solution silver particles, and/or suspension silver particles.

A feed 2804C may include any food that is known or developed in the future suitable for an adult animal (e.g., fowl, livestock, or other animal). Examples of a feed 2804C for an animal include, but are not limited to, one or more grains (e.g., corn, corn meal, soybeans, soybean meal, wheat, wheat middlings, peas, oats, etc.), fish meal, kelp, aragonite, leafy greens, beans, one or more cereals, fruits, vegetables, insects, insect meal, etc., and combinations thereof, among other food(s) that is/are suitable for an adult animal (e.g., fowl, livestock, and/or other animal).

In various embodiments, the feed 2804C can include any suitable nutrients (e.g., vitamins, minerals, supplements, etc.) specifically designed and/or tailored for an adult animal. In additional or alternative embodiments, the feed 2804C can include any suitable texture and/or size (e.g., a larger size) specifically designed and/or tailored for an adult animal.

In various embodiments, the ratio of compound 2802 including silver particles and feed 2804C is in the range of about 1:50 (1 part compound 2802 to 50 parts feed 2804C) to about 1:10 (1 part compound 2802 to 10 parts feed 2804C), among other ratios and/or ranges that are possible, each of which is contemplated herein. In certain embodiments, the ratio of compound 2802 including silver particles and feed 2804C is about 1:30 (1 part compound 2802 to 30 parts feed 2804C), among other ratios that are possible, each of which is contemplated herein.

The medicated grower food 2800B, in various embodiments, includes an amount of the compound 2802 including silver particles in the range of about 0.1 ounces to about 1 ounce per pound, by weight and/or volume. In certain embodiments, the medicated starter food 2800B includes about 0.5 ounces of the compound 2802 including silver particles per pound, by weight and/or volume.

The compound 2802 including silver particles may be combined with the feed 2804B using any suitable method and/or technique that is/are known or developed in the future. In some embodiments, the compound 2802 including silver particles is mixed with and/or into the feed 2804B to create the medicated starter food 2800B. In other embodiments, the compound 2802 including silver particles is sprayed (e.g., topically) onto the feed 2804B to create the medicated starter food 2800B. In further embodiments, the compound 2802 including silver particles is mixed with/into the feed 2804B and is also sprayed onto the feed 2804B to create the medicated starter food 2800B.

The medicated grower food 2800C may be fed to the animal when the animal reaches adulthood, which can be any amount of time and/or age of the animal. In various embodiments, the medicated grower food 2800C may be fed to, for example, a chicken beginning at day 10 of its life, among other number of days that are less than 10 days or greater than 10 days that are possible, each of which is contemplated herein.

Testing of the various embodiments of a silver compound 2802 disclosed herein shows that ingesting (e.g., oral ingestion) the various embodiments of the silver compound 2802 in food (e.g., on a daily/regular basis in food can kill the bacteria Clostridium perfringens in an animal (e.g., livestock and particularly, fowl (chickens)). In addition, testing of the various embodiments of the silver compound 2802 shows that ingesting (e.g., oral ingestion) the various embodiments of the silver compound 2802 on a daily/regular basis in food can treat and/or prevent necrotic enteritis in an animal (e.g., livestock and particularly, fowl (chickens)).

Some studies showed that daily/regular ingestion of a medicated food 2800A, starter food 2800B, and/or grower food 2800C is helpful in killing the bacteria Clostridium perfringens in the digestive system of fowl (chickens), treating necrotic enteritis in fowl (chickens), and/or preventing necrotic enteritis in fowl (chickens) to obtain some of the above benefits. In addition to killing the bacteria Clostridium perfringens in the digestive system an animal (e.g., livestock and particularly, fowl (chickens)), treating necrotic enteritis in an animal (e.g., livestock and particularly, fowl (chickens)), and/or preventing necrotic enteritis in an animal (e.g., livestock and particularly, fowl (chickens)), various tests show that ingestion (e.g., daily and/or regular ingestion) of a medicated food 2800A, medicated starter food 2800B, and/or medicated grower food 2800C results in an improvement/increase in weight gain, a reduced amount of gut lesions, elimination of gut lesions in the gut, at least a reduced mortality rate, and/or an improved feed efficiency in a challenged control group of fowl (chickens). Furthermore, various tests show that ingestion (e.g., daily and/or regular ingestion) of a medicated food 2800A, medicated starter food 2800B, and/or medicated grower food 2800C by healthy fowl (chickens) results in an improvement/increase in feed efficiency by up to three percent (3%) compared to fowl eating feed 2804A, 2804B, and/or 2804C alone (or not eating medicated food 2800A, medicated starter food 2800B, and/or medicated grower food 2800C), which is a significant improvement.

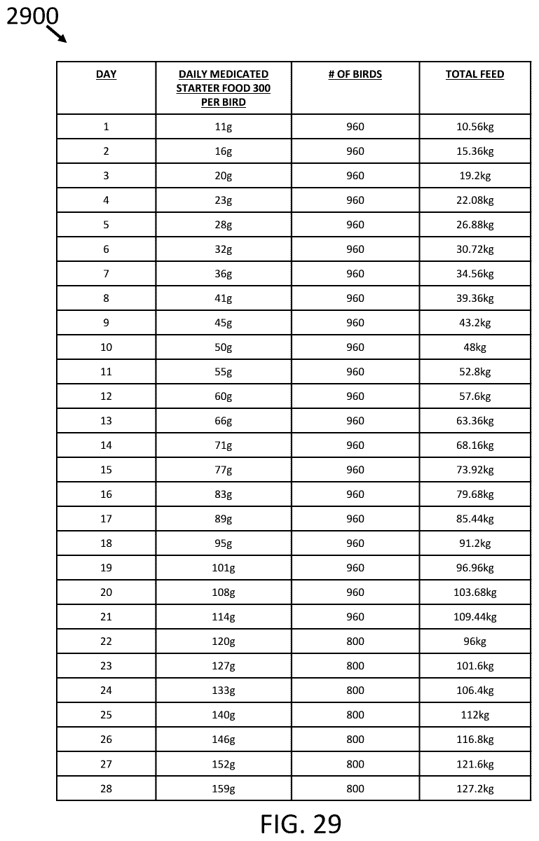

Referring to FIG. 29, FIG. 29 is a chart 2900 illustrating one embodiment of a feeding schedule for a group of chicks infected with the bacteria Clostridium perfringens to determine the amount of silver particles that is to be added to feed to create a medicated food with an amount of silver particles sufficient to kill the Clostridium perfringens, prevent necrotic enteritis in the chicks, and/or treat necrotic enteritis in the chicks. The amount of food that each chick consumed on a daily basis determined how much of the compound 2802 including silver particles needed to be combined with the feed so that each bird consumed at least 2 ppm, by weight and/or volume, of the silver particles daily.

The chart 2900 shows that as the chicks age and increase in size, they consumed more Ag particles each respective day as their daily consumption of medicated food 2800 (e.g., the compound 2802 including silver particles combined with the feed 2804) increased. For example, on day 1 each chick consumed approximately 11 grams of medicated food 2800 and the amount of medicated food 2800 consumed each day by each bird gradually increased to 159 grams of medicated food 2800 on day 28. The increase in daily ingestion of the silver particles came as a natural result of each chick growing and needing to consume more calories to sustain growth.

Based on the daily intake of at least 2 ppm, by weight and/or volume, of silver particles, it was determined that the group of chicks would have a lower mortality rate as well as a lower infection rate than the normal rates for chicks. An unexpected result found that at a 1:30 ratio of compound 2802 including silver particles to feed 2804A, 2804B, 2804C, the chicks seemed to experience an improved feed efficiency over normal rates for chicks, meaning the infected group ate less food than is normal for chicks and still gained weight consistent with the normal growth weight of chicks. While a daily ingestion rate of 2 ppm, by weight and/or volume, of silver particles was the target amount of the feeding schedule shown in the chart 2900, the various other feed schedules are possible based on the various embodiments of the medicated food 2800A, medicated starter food 2800B, and/or medicated grower food 2800C disclosed herein and such embodiments are not limited to 2 ppm, by weight and/or volume.

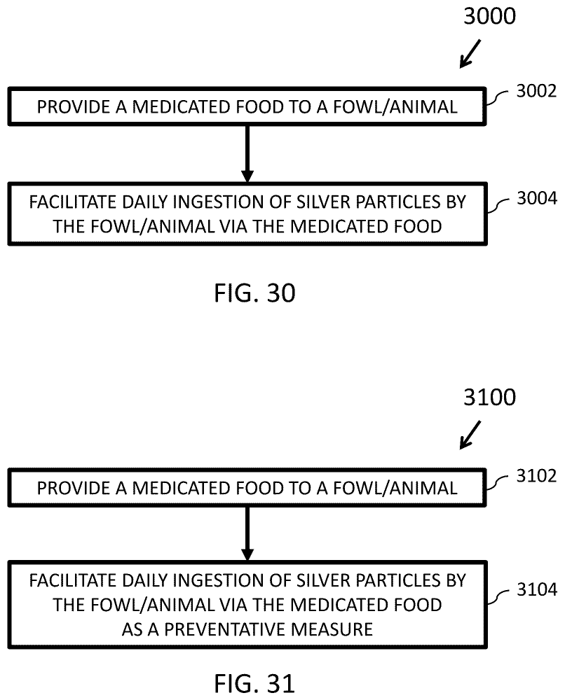

FIG. 30 is a block diagram of one embodiment of a method 3000 for using a medicated food (e.g., medicated food 2800A) to protect the digestive system of an animal (e.g., fowl, livestock, or other animal) from the bacteria Clostridium perfringens, prevent necrotic enteritis in the animal, and/or treat necrotic enteritis in the animal. At least in the illustrated embodiment, the method 3000 can begin by providing a medicated food 2800A to an animal (block 3002). The medicated food 2800A may include any embodiment of the medicated food 2800A discussed elsewhere herein.

The medicated food 2800A can be provided to the animal in any suitable manner that is known or developed in the future. Examples of providing the medicated food 2800A to the animal can include, but is not limited to, automatically and/or passively (e.g., via food trough) and/or manual administration by a caregiver (e.g., via force feeding, etc.), among other techniques/methods that are possible, each of which is contemplated herein.

The silver particles in the compound 2802 can include any embodiment of the silver particles discussed herein. That is, the silver particles may include nano-silver particles 100, colloidal silver particles, ionic silver particles, solution silver particles, and/or suspension silver particles.

In some embodiments, the compound 2802 includes an amount of silver particles (e.g., Ag particles), which can include any amount of silver particles in the range of about 0.5 ppm to about 100 ppm, by weight and/or volume, as discussed elsewhere herein. In certain embodiments, the amount of silver particles is 10 ppm or 32 ppm, by weight and/or volume, among other amounts that are possible, each of which is contemplated herein.

In various embodiments, the ratio of compound 2802 including silver particles and feed 2804A is in the range of about 1:50 to about 1:10, as discussed elsewhere herein. In certain embodiments, the ratio of compound 2802 including silver particles and feed 2804A is about 1:30, as discussed elsewhere herein.

The medicated food 2800A, in various embodiments, includes an amount of the compound 2802 including silver particles in the range of about 0.1 ounces to about 1 ounce per pound, by weight and/or volume. In certain embodiments, the medicated food 2800A includes about 0.5 ounces of the compound 2802 including silver particles per pound, by weight and/or volume.

The method 3000 further includes facilitating daily ingestion of the silver particles by the animal (block 3004). Facilitating daily ingestion of the silver particles by the animal may include feeding the medicated food 2800A to the animal. The medicated starter food 2800A may be fed to the animal in any suitable technique and/or method that encourages and/or causes an animal to ingest the silver particles on a daily and/or other similar regular basis. Examples of providing the silver compound to the animal can include, but is not limited to, automatically and/or passively (e.g., via regular feeding) and/or manual administration by a caregiver (e.g., via force feeding, etc.), among other techniques/methods that are possible, each of which is contemplated herein.

In various embodiments, providing the medicated food 2800A and facilitating daily ingestion of the silver particles is performed to kill the bacteria Clostridium perfringens in the digestive system (e.g., intestines) of the animal, and fowl in particular. In additional or alternative embodiments, providing the medicated food 2800A and facilitating daily ingestion of the silver particles is performed to prevent and/or treat necrotic enteritis in the animal, and especially in fowl.

In various embodiments, facilitating daily ingestion or causing daily ingestion of the silver particles (e.g., via manual administration) may include ingestion of the silver particles any suitable number of times a day. For example, ingesting an amount of the silver particles can include ingestion once a day, two times a day, three times a day, etc., among other quantities that are possible, each of which is contemplated herein. In certain embodiments, facilitating daily ingestion or causing daily ingestion of the silver particles may include the compound including the silver particles being added to each feed 2804A present to the animal (e.g., sprayed on the feed 2804A one or more times a day).

In at least some embodiments in which the silver particles are ingested or caused to be ingested at least twice daily, each ingestion is separated by a predetermined minimum amount of time, which can be any suitable amount of time that can facilitate protecting and/or treating the digestive system of an animal. Example predetermined minimum amounts of time can include, but are not limited to, one hour, two hours, four hours, six hours, eight hours, twelve hours, etc., among other minimum amounts of time that are possible, each of which is contemplated herein.

FIG. 31 is a block diagram of one embodiment of a method 3100 for using a medicated food (e.g., medicated food 2800A) to protect the digestive system of an animal (e.g., fowl, livestock, or other animal) from the bacteria Clostridium perfringens and/or prevent necrotic enteritis in the animal. At least in the illustrated embodiment, the method 3100 can begin by providing a medicated food 2800A to an animal (block 3102). The medicated food 2800A may include any embodiment of the medicated food 2800A discussed elsewhere herein.

The medicated food 2800A can be provided to the animal in any suitable manner that is known or developed in the future. Examples of providing the medicated food 2800A to the animal can include, but is not limited to, automatically and/or passively (e.g., via food trough) and/or manual administration by a caregiver (e.g., via force feeding, etc.), among other techniques/methods that are possible, each of which is contemplated herein.

The silver particles in the compound 2802 can include any embodiment of the silver particles discussed herein. That is, the silver particles may include nano-silver particles 100, colloidal silver particles, ionic silver particles, solution silver particles, and/or suspension silver particles.

In some embodiments, the compound 2802 includes an amount of silver particles (e.g., Ag particles), which can include any amount of silver particles in the range of about 0.5 ppm to about 100 ppm, by weight and/or volume, as discussed elsewhere herein. In certain embodiments, the amount of silver particles is 10 ppm or 32 ppm, by weight and/or volume, among other amounts that are possible, each of which is contemplated herein.

In various embodiments, the ratio of compound 2802 including silver particles and feed 2804A is in the range of about 1:50 to about 1:10, as discussed elsewhere herein. In certain embodiments, the ratio of compound 2802 including silver particles and feed 2804A is about 1:30, as discussed elsewhere herein.

The medicated food 2800A, in various embodiments, includes an amount of the compound 2802 including silver particles in the range of about 0.1 ounces to about 1 ounce per pound, by weight and/or volume. In certain embodiments, the medicated food 2800A includes about 0.5 ounces of the compound 2802 including silver particles per pound, by weight and/or volume.

The method 3100 further includes facilitating daily ingestion of the silver particles by the animal as a preventative measure to kill Clostridium perfringens in the animal and/or prevent necrotic enteritis in the animal (block 3104). Facilitating daily ingestion of the silver particles by the animal may include feeding the medicated food 2800A to the animal. The medicated starter food 2800A may be fed to the animal in any suitable technique and/or method that encourages and/or causes an animal to ingest the silver particles on a daily and/or other similar regular basis. Examples of providing the silver compound to the animal can include, but is not limited to, automatically and/or passively (e.g., via regular feeding) and/or manual administration by a caregiver (e.g., via force feeding, etc.), among other techniques/methods that are possible, each of which is contemplated herein.

In various embodiments, providing the medicated food 2800A and facilitating daily ingestion of the silver particles is performed to kill the bacteria Clostridium perfringens in the digestive system (e.g., intestines) of the animal, and fowl in particular. In additional or alternative embodiments, providing the medicated food 2800A and facilitating daily ingestion of the silver particles is performed to prevent necrotic enteritis in the animal, and especially in fowl.

In various embodiments, facilitating daily ingestion or causing daily ingestion of the silver particles (e.g., via manual administration) may include ingestion of the silver particles any suitable number of times a day. For example, ingesting an amount of the silver particles can include ingestion once a day, two times a day, three times a day, etc., among other quantities that are possible, each of which is contemplated herein. In certain embodiments, facilitating daily ingestion or causing daily ingestion of the silver particles may include the compound including the silver particles being added to each feed 2804A present to the animal (e.g., sprayed on the feed 2804A one or more times a day).

In at least some embodiments in which the silver particles are ingested or caused to be ingested at least twice daily, each ingestion is separated by a predetermined minimum amount of time, which can be any suitable amount of time that can facilitate protecting and/or treating the digestive system of an animal. Example predetermined minimum amounts of time can include, but are not limited to, one hour, two hours, four hours, six hours, eight hours, twelve hours, etc., among other minimum amounts of time that are possible, each of which is contemplated herein.

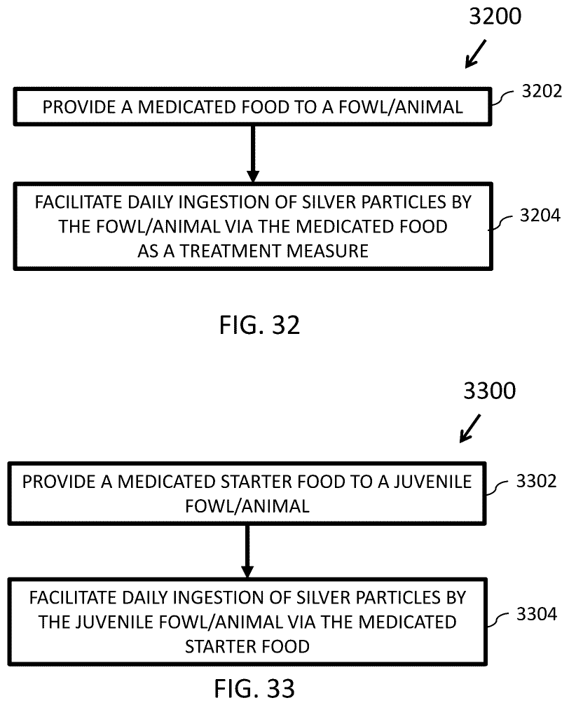

FIG. 32 is a block diagram of one embodiment of a method 3200 for using a medicated food (e.g., medicated food 2800A) to protect the digestive system of an animal (e.g., fowl, livestock, or other animal) from the bacteria Clostridium perfringens and/or treat necrotic enteritis in the animal. At least in the illustrated embodiment, the method 3200 can begin by providing a medicated food 2800A to an animal (block 3202). The medicated food 2800A may include any embodiment of the medicated food 2800A discussed elsewhere herein.

The medicated food 2800A can be provided to the animal in any suitable manner that is known or developed in the future. Examples of providing the medicated food 2800A to the animal can include, but is not limited to, automatically and/or passively (e.g., via food trough) and/or manual administration by a caregiver (e.g., via force feeding, etc.), among other techniques/methods that are possible, each of which is contemplated herein.

The silver particles in the compound 2802 can include any embodiment of the silver particles discussed herein. That is, the silver particles may include nano-silver particles 100, colloidal silver particles, ionic silver particles, solution silver particles, and/or suspension silver particles.

In some embodiments, the compound 2802 includes an amount of silver particles (e.g., Ag particles), which can include any amount of silver particles in the range of about 0.5 ppm to about 100 ppm, by weight and/or volume, as discussed elsewhere herein. In certain embodiments, the amount of silver particles is 10 ppm or 32 ppm, by weight and/or volume, among other amounts that are possible, each of which is contemplated herein.

In various embodiments, the ratio of compound 2802 including silver particles and feed 2804B is in the range of about 1:50 to about 1:10, as discussed elsewhere herein. In certain embodiments, the ratio of compound 2802 including silver particles and feed 2804B is about 1:30, as discussed elsewhere herein.

The medicated starter food 2800B, in various embodiments, includes an amount of the compound 2802 including silver particles in the range of about 0.1 ounces to about 1 ounce per pound, by weight and/or volume. In certain embodiments, the medicated starter food 2800B includes about 0.5 ounces of the compound 2802 including silver particles per pound, by weight and/or volume.

The method 3200 further includes facilitating daily ingestion of the silver particles by the juvenile animal as a treatment measure to kill Clostridium perfringens in the juvenile animal and/or treat necrotic enteritis in the juvenile animal (block 3204). Facilitating daily ingestion of the silver particles by the animal may include feeding the medicated food 2800A to the animal. The medicated starter food 2800A may be fed to the animal in any suitable technique and/or method that encourages and/or causes an animal to ingest the silver particles on a daily and/or other similar regular basis. Examples of providing the silver compound to the animal can include, but is not limited to, automatically and/or passively (e.g., via regular feeding) and/or manual administration by a caregiver (e.g., via force feeding, etc.), among other techniques/methods that are possible, each of which is contemplated herein.

In various embodiments, providing the medicated food 2800A and facilitating daily ingestion of the silver particles is performed to kill the bacteria Clostridium perfringens in the digestive system (e.g., intestines) of the animal, and fowl in particular. In additional or alternative embodiments, providing the medicated food 2800A and facilitating daily ingestion of the silver particles is performed to treat necrotic enteritis in the animal, and especially in fowl.

In various embodiments, facilitating daily ingestion or causing daily ingestion of the silver particles (e.g., via manual administration) may include ingestion of the silver particles any suitable number of times a day. For example, ingesting an amount of the silver particles can include ingestion once a day, two times a day, three times a day, etc., among other quantities that are possible, each of which is contemplated herein. In certain embodiments, facilitating daily ingestion or causing daily ingestion of the silver particles may include the compound including the silver particles being added to each feed 2804A present to the animal (e.g., sprayed on the feed 2804A one or more times a day).

In at least some embodiments in which the silver particles are ingested or caused to be ingested at least twice daily, each ingestion is separated by a predetermined minimum amount of time, which can be any suitable amount of time that can facilitate protecting and/or treating the digestive system of an animal. Example predetermined minimum amounts of time can include, but are not limited to, one hour, two hours, four hours, six hours, eight hours, twelve hours, etc., among other minimum amounts of time that are possible, each of which is contemplated herein.

While the various embodiments of methods 3000, 3100, and 3200 in FIGS. 30, 31, and 32, respectively, are disclosed with reference to an animal (e.g., fowl, livestock, or other animal), the various embodiments of methods 3000, 3100, and 3200 are not limited to the example types of animals. That is, the various embodiments of methods 3000, 3100, and/or 3200 can be specifically applied to chickens, ducks, turkeys, geese, pigs, cattle, sheep, goats, horses, etc., among other fowl and/or livestock that are possible, each of which is contemplated herein. In addition, various other embodiments of methods 3000, 3100, and/or 3200 include protecting the digestive system of any other animal and/or type of animal.

FIG. 33 is a block diagram of one embodiment of a method 3300 for using a medicated starter food (e.g., medicated starter food 2800B) to protect the digestive system of a juvenile animal (e.g., fowl, livestock, or other animal) from the bacteria Clostridium perfringens, prevent necrotic enteritis in the juvenile animal, and/or treat necrotic enteritis in the juvenile animal. At least in the illustrated embodiment, the method 3300 can begin by providing a medicated starter food 2800B to a juvenile animal (block 3302). The medicated starter food 2800B may include any embodiment of the medicated starter food 2800B discussed elsewhere herein.

The medicated starter food 2800B can be provided to the juvenile animal in any suitable manner that is known or developed in the future. Examples of providing the medicated starter food 2800B to the juvenile animal can include, but is not limited to, automatically and/or passively (e.g., via food trough) and/or manual administration by a caregiver (e.g., via force feeding, etc.), among other techniques/methods that are possible, each of which is contemplated herein.

The silver particles in the compound 2802 can include any embodiment of the silver particles discussed herein. That is, the silver particles may include nano-silver particles 100, colloidal silver particles, ionic silver particles, solution silver particles, and/or suspension silver particles.

In some embodiments, the compound 2802 includes an amount of silver particles (e.g., Ag particles), which can include any amount of silver particles in the range of about 0.5 ppm to about 100 ppm, by weight and/or volume, as discussed elsewhere herein. In certain embodiments, the amount of silver particles is 10 ppm or 32 ppm, by weight and/or volume, among other amounts that are possible, each of which is contemplated herein.

In various embodiments, the ratio of compound 2802 including silver particles and feed 2804B is in the range of about 1:50 to about 1:10, as discussed elsewhere herein. In certain embodiments, the ratio of compound 2802 including silver particles and feed 2804B is about 1:30, as discussed elsewhere herein.

The medicated starter food 2800B, in various embodiments, includes an amount of the compound 2802 including silver particles in the range of about 0.1 ounces to about 1 ounce per pound, by weight and/or volume. In certain embodiments, the medicated starter food 2800B includes about 0.5 ounces of the compound 2802 including silver particles per pound, by weight and/or volume.

The method 3300 further includes facilitating daily ingestion of the silver particles by the juvenile animal (block 3304). Facilitating daily ingestion of the silver particles by the juvenile animal may include feeding the medicated starter food 2800B to the juvenile animal. The medicated starter food 2800B may be fed to the juvenile animal in any suitable technique and/or method that encourages and/or causes a juvenile animal to ingest the silver particles on a daily and/or other similar regular basis. Examples of providing the silver compound to the juvenile animal can include, but is not limited to, automatically and/or passively (e.g., via regular feeding) and/or manual administration by a caregiver (e.g., via force feeding, etc.), among other techniques/methods that are possible, each of which is contemplated herein.

In various embodiments, providing the medicated starter food 2800B and facilitating daily ingestion of the silver particles is performed to kill the bacteria Clostridium perfringens in the digestive system (e.g., intestines) of the juvenile animal, and juvenile fowl in particular. In additional or alternative embodiments, providing the medicated starter food 2800B and facilitating daily ingestion of the silver particles is performed to prevent and/or treat necrotic enteritis in the juvenile animal, and especially in juvenile fowl.

In various embodiments, facilitating daily ingestion or causing daily ingestion of the silver particles (e.g., via manual administration) may include ingestion of the silver particles any suitable number of times a day. For example, ingesting an amount of the silver particles can include ingestion once a day, two times a day, three times a day, etc., among other quantities that are possible, each of which is contemplated herein. In certain embodiments, facilitating daily ingestion or causing daily ingestion of the silver particles may include the compound including the silver particles being added to each feed 2804B present to the animal (e.g., sprayed on the feed 2804B one or more times a day).

In at least some embodiments in which the silver particles are ingested or caused to be ingested at least twice daily, each ingestion is separated by a predetermined minimum amount of time, which can be any suitable amount of time that can facilitate protecting and/or treating the digestive system of an animal. Example predetermined minimum amounts of time can include, but are not limited to, one hour, two hours, four hours, six hours, eight hours, twelve hours, etc., among other minimum amounts of time that are possible, each of which is contemplated herein.

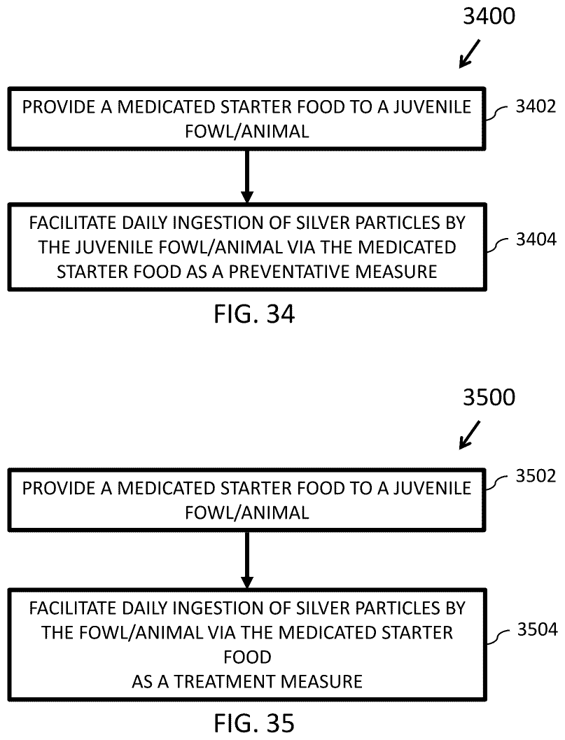

FIG. 34 is a block diagram of one embodiment of a method 3400 for using a medicated starter food (e.g., medicated starter food 2800B) to protect the digestive system of a juvenile animal (e.g., fowl, livestock, or other animal) from the bacteria Clostridium perfringens and/or prevent necrotic enteritis in the juvenile animal. At least in the illustrated embodiment, the method 3400 can begin by providing a medicated starter food 2800B to a juvenile animal (block 3402). The medicated starter food 2800B may include any embodiment of the medicated starter food 2800B discussed elsewhere herein.

The medicated starter food 2800B can be provided to the juvenile animal in any suitable manner that is known or developed in the future. Examples of providing the medicated starter food 2800B to the juvenile animal can include, but is not limited to, automatically and/or passively (e.g., via food trough) and/or manual administration by a caregiver (e.g., via force feeding, etc.), among other techniques/methods that are possible, each of which is contemplated herein.

The silver particles in the compound 2802 can include any embodiment of the silver particles discussed herein. That is, the silver particles may include nano-silver particles 100, colloidal silver particles, ionic silver particles, solution silver particles, and/or suspension silver particles.

In some embodiments, the compound 2802 includes an amount of silver particles (e.g., Ag particles), which can include any amount of silver particles in the range of about 0.5 ppm to about 100 ppm, by weight and/or volume, as discussed elsewhere herein. In certain embodiments, the amount of silver particles is 10 ppm or 32 ppm, by weight and/or volume, among other amounts that are possible, each of which is contemplated herein.

In various embodiments, the ratio of compound 2802 including silver particles and feed 2804B is in the range of about 1:50 to about 1:10, as discussed elsewhere herein. In certain embodiments, the ratio of compound 2802 including silver particles and feed 2804B is about 1:30, as discussed elsewhere herein.

The medicated starter food 2800B, in various embodiments, includes an amount of the compound 2802 including silver particles in the range of about 0.1 ounces to about 1 ounce per pound, by weight and/or volume. In certain embodiments, the medicated starter food 2800B includes about 0.5 ounces of the compound 2802 including silver particles per pound, by weight and/or volume.

The method 3400 further includes facilitating daily ingestion of the silver particles by the juvenile animal as a preventative measure to kill Clostridium perfringens in the juvenile animal and/or prevent necrotic enteritis in the juvenile animal (block 3404). Facilitating daily ingestion of the silver particles by the animal may include feeding the medicated starter food 2800B to the juvenile animal. The medicated starter food 2800B may be fed to the juvenile animal in any suitable technique and/or method that encourages and/or causes a juvenile animal to ingest the silver particles on a daily and/or other similar regular basis. Examples of providing the silver compound to the juvenile animal can include, but is not limited to, automatically and/or passively (e.g., via regular feeding) and/or manual administration by a caregiver (e.g., via force feeding, etc.), among other techniques/methods that are possible, each of which is contemplated herein.

In various embodiments, providing the medicated starter food 2800B and facilitating daily ingestion of the silver particles is performed to kill the bacteria Clostridium perfringens in the digestive system (e.g., intestines) of the juvenile animal, and juvenile fowl in particular. In additional or alternative embodiments, providing the medicated starter food 2800B and facilitating daily ingestion of the silver particles is performed to prevent necrotic enteritis in the juvenile animal, and especially in juvenile fowl.

In various embodiments, facilitating daily ingestion or causing daily ingestion of the silver particles (e.g., via manual administration) may include ingestion of the silver particles any suitable number of times a day. For example, ingesting an amount of the silver particles can include ingestion once a day, two times a day, three times a day, etc., among other quantities that are possible, each of which is contemplated herein. In certain embodiments, facilitating daily ingestion or causing daily ingestion of the silver particles may include the compound including the silver particles being added to each feed 2804B present to the juvenile animal (e.g., sprayed on the feed 2804B one or more times a day).

In at least some embodiments in which the silver particles are ingested or caused to be ingested at least twice daily, each ingestion is separated by a predetermined minimum amount of time, which can be any suitable amount of time that can facilitate protecting and/or treating the digestive system of an animal. Example predetermined minimum amounts of time can include, but are not limited to, one hour, two hours, four hours, six hours, eight hours, twelve hours, etc., among other minimum amounts of time that are possible, each of which is contemplated herein.

FIG. 35 is a block diagram of one embodiment of a method 3500 for using a medicated starter food (e.g., medicated starter food 2800B) to protect the digestive system of a juvenile animal (e.g., fowl, livestock, or other animal) from the bacteria Clostridium perfringens and/or treat necrotic enteritis in the juvenile animal. At least in the illustrated embodiment, the method 3500 can begin by providing a medicated starter food 2800B to an animal (block 3502). The medicated starter food 2800B may include any embodiment of the medicated starter food 2800B discussed elsewhere herein.

The medicated starter food 2800B can be provided to the animal in any suitable manner that is known or developed in the future. Examples of providing the medicated starter food 2800B to the juvenile animal can include, but is not limited to, automatically and/or passively (e.g., via food trough) and/or manual administration by a caregiver (e.g., via force feeding, etc.), among other techniques/methods that are possible, each of which is contemplated herein.

The silver particles in the compound 2802 can include any embodiment of the silver particles discussed herein. That is, the silver particles may include nano-silver particles 100, colloidal silver particles, ionic silver particles, solution silver particles, and/or suspension silver particles.

In some embodiments, the compound 2802 includes an amount of silver particles (e.g., Ag particles), which can include any amount of silver particles in the range of about 0.5 ppm to about 100 ppm, by weight and/or volume, as discussed elsewhere herein. In certain embodiments, the amount of silver particles is 10 ppm or 32 ppm, by weight and/or volume, among other amounts that are possible, each of which is contemplated herein.

In various embodiments, the ratio of compound 2802 including silver particles and feed 2804B is in the range of about 1:50 to about 1:10, as discussed elsewhere herein. In certain embodiments, the ratio of compound 2802 including silver particles and feed 2804B is about 1:30, as discussed elsewhere herein.

The medicated starter food 2800B, in various embodiments, includes an amount of the compound 2802 including silver particles in the range of about 0.1 ounces to about 1 ounce per pound, by weight and/or volume. In certain embodiments, the medicated starter food 2800B includes about 0.5 ounces of the compound 2802 including silver particles per pound, by weight and/or volume.

The method 3500 further includes facilitating daily ingestion of the silver particles by the animal as a treatment measure to kill Clostridium perfringens in the juvenile animal and/or treat necrotic enteritis in the juvenile animal (block 3504). Facilitating daily ingestion of the silver particles by the juvenile animal may include feeding the medicated starter food 2800B to the juvenile animal. The medicated starter food 2800B may be fed to the juvenile animal in any suitable technique and/or method that encourages and/or causes a juvenile animal to ingest the silver particles on a daily and/or other similar regular basis. Examples of providing the silver compound to the juvenile animal can include, but is not limited to, automatically and/or passively (e.g., via regular feeding) and/or manual administration by a caregiver (e.g., via force feeding, etc.), among other techniques/methods that are possible, each of which is contemplated herein.

In various embodiments, providing the medicated starter food 2800B and facilitating daily ingestion of the silver particles is performed to kill the bacteria Clostridium perfringens in the digestive system (e.g., intestines) of the juvenile animal, and juvenile fowl in particular. In additional or alternative embodiments, providing the medicated starter food 2800B and facilitating daily ingestion of the silver particles is performed to treat necrotic enteritis in the animal, and especially in juvenile fowl.

In various embodiments, facilitating daily ingestion or causing daily ingestion of the silver particles (e.g., via manual administration) may include ingestion of the silver particles any suitable number of times a day. For example, ingesting an amount of the silver particles can include ingestion once a day, two times a day, three times a day, etc., among other quantities that are possible, each of which is contemplated herein. In certain embodiments, facilitating daily ingestion or causing daily ingestion of the silver particles may include the compound including the silver particles being added to each feed 2804B present to the animal (e.g., sprayed on the feed 2804B one or more times a day).

In at least some embodiments in which the silver particles are ingested or caused to be ingested at least twice daily, each ingestion is separated by a predetermined minimum amount of time, which can be any suitable amount of time that can facilitate protecting and/or treating the digestive system of an animal. Example predetermined minimum amounts of time can include, but are not limited to, one hour, two hours, four hours, six hours, eight hours, twelve hours, etc., among other minimum amounts of time that are possible, each of which is contemplated herein.

While the various embodiments of methods 3300, 3400, and 3500 in FIGS. 33, 34, and 35, respectively, are disclosed with reference to a juvenile animal (e.g., juvenile fowl, juvenile livestock, or other juvenile animal), the various embodiments of methods 3300, 3400, and 3500 are not limited to the example types of animals. That is, the various embodiments of methods 3300, 3400, and/or 3500 can be specifically applied to chickens, ducks, turkeys, geese, pigs, cattle, sheep, goats, horses, etc., among other fowl and/or livestock that are possible, each of which is contemplated herein. In addition, various other embodiments of methods 3300, 3400, and/or 3500 include protecting the digestive system of any other animal and/or type of animal.

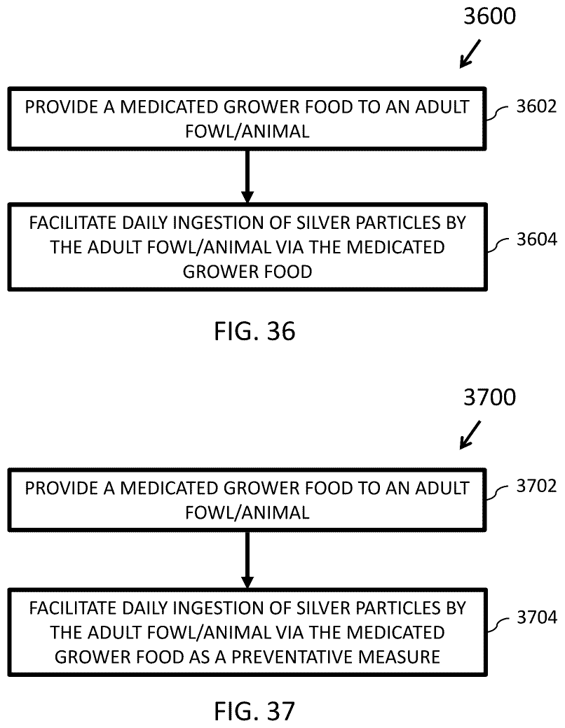

FIG. 36 is a block diagram of one embodiment of a method 3600 for using a medicated grower food (e.g., medicated grower food 2800C) to protect the digestive system of an adult animal (e.g., fowl, livestock, or other animal) from the bacteria Clostridium perfringens, prevent necrotic enteritis in the adult animal, and/or treat necrotic enteritis in the adult animal. At least in the illustrated embodiment, the method 3600 can begin by providing a medicated grower food 2800C to an adult animal (block 3602). The medicated grower food 2800C may include any embodiment of the medicated grower food 2800C discussed elsewhere herein.

The medicated grower food 2800C can be provided to the adult animal in any suitable manner that is known or developed in the future. Examples of providing the medicated grower food 2800C to the adult animal can include, but is not limited to, automatically and/or passively (e.g., via food trough) and/or manual administration by a caregiver (e.g., via force feeding, etc.), among other techniques/methods that are possible, each of which is contemplated herein.

The silver particles in the compound 2802 can include any embodiment of the silver particles discussed herein. That is, the silver particles may include nano-silver particles 100, colloidal silver particles, ionic silver particles, solution silver particles, and/or suspension silver particles.

In some embodiments, the compound 2802 includes an amount of silver particles (e.g., Ag particles), which can include any amount of silver particles in the range of about 0.5 ppm to about 100 ppm, by weight and/or volume, as discussed elsewhere herein. In certain embodiments, the amount of silver particles is 10 ppm or 32 ppm, by weight and/or volume, among other amounts that are possible, each of which is contemplated herein.

In various embodiments, the ratio of compound 2802 including silver particles and feed 2804C is in the range of about 1:50 to about 1:10, as discussed elsewhere herein. In certain embodiments, the ratio of compound 2802 including silver particles and feed 2804C is about 1:30, as discussed elsewhere herein.

The medicated grower food 2800C, in various embodiments, includes an amount of the compound 2802 including silver particles in the range of about 0.1 ounces to about 1 ounce per pound, by weight and/or volume. In certain embodiments, the medicated starter food 2800C includes about 0.5 ounces of the compound 2802 including silver particles per pound, by weight and/or volume.

The method 3600 further includes facilitating daily ingestion of the silver particles by the adult animal (block 3604). Facilitating daily ingestion of the silver particles by the adult animal may include feeding the medicated grower food 2800C to the adult animal. The medicated grower food 2800C may be fed to the adult animal in any suitable technique and/or method that encourages and/or causes an adult animal to ingest the silver particles on a daily and/or other similar regular basis. Examples of providing the silver compound to the adult animal can include, but is not limited to, automatically and/or passively (e.g., via regular feeding) and/or manual administration by a caregiver (e.g., via force feeding, etc.), among other techniques/methods that are possible, each of which is contemplated herein.

In various embodiments, providing the medicated grower food 2800C and facilitating daily ingestion of the silver particles is performed to kill the bacteria Clostridium perfringens in the digestive system (e.g., intestines) of the adult animal, and adult fowl in particular. In additional or alternative embodiments, providing the medicated grower food 2800C and facilitating daily ingestion of the silver particles is performed to prevent and/or treat necrotic enteritis in the adult animal, and especially in adult fowl.

In various embodiments, facilitating daily ingestion or causing daily ingestion of the silver particles (e.g., via manual administration) may include ingestion of the silver particles any suitable number of times a day. For example, ingesting an amount of the silver particles can include ingestion once a day, two times a day, three times a day, etc., among other quantities that are possible, each of which is contemplated herein. In certain embodiments, facilitating daily ingestion or causing daily ingestion of the silver particles may include the compound including the silver particles being added to each feed 2804C present to the animal (e.g., sprayed on the feed 2804C one or more times a day).

In at least some embodiments in which the silver particles are ingested or caused to be ingested at least twice daily, each ingestion is separated by a predetermined minimum amount of time, which can be any suitable amount of time that can facilitate protecting and/or treating the digestive system of an animal. Example predetermined minimum amounts of time can include, but are not limited to, one hour, two hours, four hours, six hours, eight hours, twelve hours, etc., among other minimum amounts of time that are possible, each of which is contemplated herein.

FIG. 37 is a block diagram of one embodiment of a method 3700 for using a medicated grower food (e.g., medicated grower food 2800C) to protect the digestive system of an adult animal (e.g., fowl, livestock, or other animal) from the bacteria Clostridium perfringens and/or prevent necrotic enteritis in the adult animal. At least in the illustrated embodiment, the method 3700 can begin by providing a medicated grower food 2800C to an adult animal (block 3702). The medicated grower food 2800C may include any embodiment of the medicated grower food 2800C discussed elsewhere herein.

The medicated grower food 2800C can be provided to the adult animal in any suitable manner that is known or developed in the future. Examples of providing the medicated grower food 2800C to the adult animal can include, but is not limited to, automatically and/or passively (e.g., via food trough) and/or manual administration by a caregiver (e.g., via force feeding, etc.), among other techniques/methods that are possible, each of which is contemplated herein.

The silver particles in the compound 2802 can include any embodiment of the silver particles discussed herein. That is, the silver particles may include nano-silver particles 100, colloidal silver particles, ionic silver particles, solution silver particles, and/or suspension silver particles.

In some embodiments, the compound 2802 includes an amount of silver particles (e.g., Ag particles), which can include any amount of silver particles in the range of about 0.5 ppm to about 100 ppm, by weight and/or volume, as discussed elsewhere herein. In certain embodiments, the amount of silver particles is 10 ppm or 32 ppm, by weight and/or volume, among other amounts that are possible, each of which is contemplated herein.

In various embodiments, the ratio of compound 2802 including silver particles and feed 2804B is in the range of about 1:50 to about 1:10, as discussed elsewhere herein. In certain embodiments, the ratio of compound 2802 including silver particles and feed 2804C is about 1:30, as discussed elsewhere herein.

The medicated grower food 2800C, in various embodiments, includes an amount of the compound 2802 including silver particles in the range of about 0.1 ounces to about 1 ounce per pound, by weight and/or volume. In certain embodiments, the medicated grower food 2800C includes about 0.5 ounces of the compound 2802 including silver particles per pound, by weight and/or volume.

The method 3700 further includes facilitating daily ingestion of the silver particles by the adult animal as a preventative measure to kill Clostridium perfringens in the adult animal and/or prevent necrotic enteritis in the adult animal (block 3704). Facilitating daily ingestion of the silver particles by the animal may include feeding the medicated grower food 2800C to the adult animal. The medicated grower food 2800C may be fed to the adult animal in any suitable technique and/or method that encourages and/or causes a adult animal to ingest the silver particles on a daily and/or other similar regular basis. Examples of providing the silver compound to the adult animal can include, but is not limited to, automatically and/or passively (e.g., via regular feeding) and/or manual administration by a caregiver (e.g., via force feeding, etc.), among other techniques/methods that are possible, each of which is contemplated herein.

In various embodiments, providing the medicated grower food 2800C and facilitating daily ingestion of the silver particles is performed to kill the bacteria Clostridium perfringens in the digestive system (e.g., intestines) of the adult animal, and adult fowl in particular. In additional or alternative embodiments, providing the medicated grower food 2800C and facilitating daily ingestion of the silver particles is performed to prevent necrotic enteritis in the adult animal, and especially in adult fowl.

In various embodiments, facilitating daily ingestion or causing daily ingestion of the silver particles (e.g., via manual administration) may include ingestion of the silver particles any suitable number of times a day. For example, ingesting an amount of the silver particles can include ingestion once a day, two times a day, three times a day, etc., among other quantities that are possible, each of which is contemplated herein. In certain embodiments, facilitating daily ingestion or causing daily ingestion of the silver particles may include the compound including the silver particles being added to each feed 2804C present to the juvenile animal (e.g., sprayed on the feed 2804C one or more times a day).

In at least some embodiments in which the silver particles are ingested or caused to be ingested at least twice daily, each ingestion is separated by a predetermined minimum amount of time, which can be any suitable amount of time that can facilitate protecting and/or treating the digestive system of an animal. Example predetermined minimum amounts of time can include, but are not limited to, one hour, two hours, four hours, six hours, eight hours, twelve hours, etc., among other minimum amounts of time that are possible, each of which is contemplated herein.

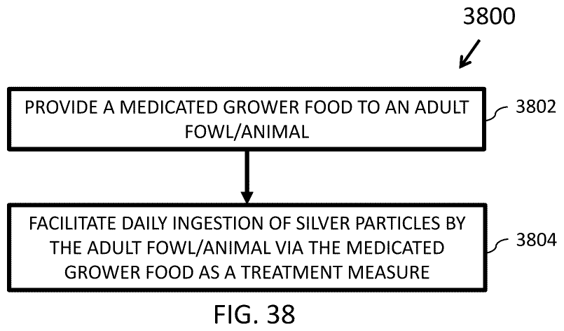

FIG. 38 is a block diagram of one embodiment of a method 3800 for using a medicated starter food (e.g., medicated grower food 2800C) to protect the digestive system of an adult animal (e.g., fowl, livestock, or other animal) from the bacteria Clostridium perfringens and/or treat necrotic enteritis in the adult animal. At least in the illustrated embodiment, the method 3800 can begin by providing a medicated grower food 2800C to an animal (block 3802). The medicated grower food 2800C may include any embodiment of the medicated grower food 2800C discussed elsewhere herein.

The medicated grower food 2800C can be provided to the animal in any suitable manner that is known or developed in the future. Examples of providing the medicated grower food 2800C to the adult animal can include, but is not limited to, automatically and/or passively (e.g., via food trough) and/or manual administration by a caregiver (e.g., via force feeding, etc.), among other techniques/methods that are possible, each of which is contemplated herein.

The silver particles in the compound 2802 can include any embodiment of the silver particles discussed herein. That is, the silver particles may include nano-silver particles 100, colloidal silver particles, ionic silver particles, solution silver particles, and/or suspension silver particles.

In some embodiments, the compound 2802 includes an amount of silver particles (e.g., Ag particles), which can include any amount of silver particles in the range of about 0.5 ppm to about 100 ppm, by weight and/or volume, as discussed elsewhere herein. In certain embodiments, the amount of silver particles is 10 ppm or 32 ppm, by weight and/or volume, among other amounts that are possible, each of which is contemplated herein.

In various embodiments, the ratio of compound 2802 including silver particles and feed 2804C is in the range of about 1:50 to about 1:10, as discussed elsewhere herein. In certain embodiments, the ratio of compound 2802 including silver particles and feed 2804C is about 1:30, as discussed elsewhere herein.

The medicated grower food 2800C, in various embodiments, includes an amount of the compound 2802 including silver particles in the range of about 0.1 ounces to about 1 ounce per pound, by weight and/or volume. In certain embodiments, the medicated starter food 2800C includes about 0.5 ounces of the compound 2802 including silver particles per pound, by weight and/or volume.

The method 3800 further includes facilitating daily ingestion of the silver particles by the animal as a treatment measure to kill Clostridium perfringens in the adult animal and/or treat necrotic enteritis in the adult animal (block 3804). Facilitating daily ingestion of the silver particles by the animal may include feeding the medicated grower food 2800C to the adult animal. The medicated grower food 2800C may be fed to the adult animal in any suitable technique and/or method that encourages and/or causes an adult animal to ingest the silver particles on a daily and/or other similar regular basis. Examples of providing the silver compound to the adult animal can include, but is not limited to, automatically and/or passively (e.g., via regular feeding) and/or manual administration by a caregiver (e.g., via force feeding, etc.), among other techniques/methods that are possible, each of which is contemplated herein.

In various embodiments, providing the medicated starter food 2800B and facilitating daily ingestion of the silver particles is performed to kill the bacteria Clostridium perfringens in the digestive system (e.g., intestines) of the adult animal, and adult fowl in particular. In additional or alternative embodiments, providing the medicated grower food 2800C and facilitating daily ingestion of the silver particles is performed to treat necrotic enteritis in the adult animal, and especially in adult fowl.

In various embodiments, facilitating daily ingestion or causing daily ingestion of the silver particles (e.g., via manual administration) may include ingestion of the silver particles any suitable number of times a day. For example, ingesting an amount of the silver particles can include ingestion once a day, two times a day, three times a day, etc., among other quantities that are possible, each of which is contemplated herein. In certain embodiments, facilitating daily ingestion or causing daily ingestion of the silver particles may include the compound including the silver particles being added to each feed 2804C present to the animal (e.g., sprayed on the feed 2804C one or more times a day).

In at least some embodiments in which the silver particles are ingested or caused to be ingested at least twice daily, each ingestion is separated by a predetermined minimum amount of time, which can be any suitable amount of time that can facilitate protecting and/or treating the digestive system of an animal. Example predetermined minimum amounts of time can include, but are not limited to, one hour, two hours, four hours, six hours, eight hours, twelve hours, etc., among other minimum amounts of time that are possible, each of which is contemplated herein.

While the various embodiments of methods 3600, 3700, and 3800 in FIGS. 36, 37, and 38, respectively, are disclosed with reference to an adult animal (e.g., adult fowl, adult livestock, or other adult animal), the various embodiments of methods 3600, 3700, and 3800 are not limited to the example types of animals. That is, the various embodiments of methods 3600, 3700, and/or 3800 can be specifically applied to chickens, ducks, turkeys, geese, pigs, cattle, sheep, goats, horses, etc., among other fowl and/or livestock that are possible, each of which is contemplated herein. In addition, various other embodiments of methods 3600, 3700, and 3800 include protecting the digestive system of any other animal and/or type of animal.

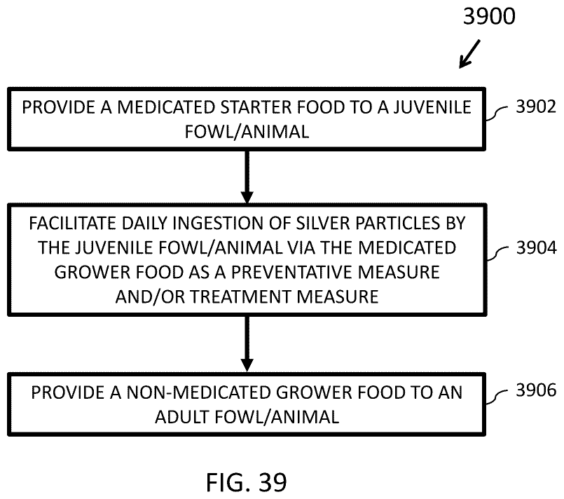

FIG. 39 is a block diagram of one embodiment of a method 3900 for using a medicated starter food (e.g., medicated starter food 2800B) to protect the digestive system of a juvenile animal (e.g., fowl, livestock, or other animal) from the bacteria Clostridium perfringens, prevent necrotic enteritis in the juvenile animal, and/or treat necrotic enteritis in the juvenile animal and using a non-medicated feed (e.g., feed 2804C) when the animal reaches adulthood. At least in the illustrated embodiment, the method 3900 can begin by providing a medicated starter food 2800B to a juvenile animal (block 3902). The medicated starter food 2800B may include any embodiment of the medicated starter food 2800B discussed elsewhere herein.

The medicated starter food 2800B can be provided to the juvenile animal in any suitable manner that is known or developed in the future. Examples of providing the medicated starter food 2800B to the juvenile animal can include, but is not limited to, automatically and/or passively (e.g., via food trough) and/or manual administration by a caregiver (e.g., via force feeding, etc.), among other techniques/methods that are possible, each of which is contemplated herein.

The silver particles in the compound 2802 can include any embodiment of the silver particles discussed herein. That is, the silver particles may include nano-silver particles 100, colloidal silver particles, ionic silver particles, solution silver particles, and/or suspension silver particles.

In some embodiments, the compound 2802 includes an amount of silver particles (e.g., Ag particles), which can include any amount of silver particles in the range of about 0.5 ppm to about 100 ppm, by weight and/or volume, as discussed elsewhere herein. In certain embodiments, the amount of silver particles is 10 ppm or 32 ppm, by weight and/or volume, among other amounts that are possible, each of which is contemplated herein.

In various embodiments, the ratio of compound 2802 including silver particles and feed 2804B is in the range of about 1:50 to about 1:10, as discussed elsewhere herein. In certain embodiments, the ratio of compound 2802 including silver particles and feed 2804B is about 1:30, as discussed elsewhere herein.

The medicated starter food 2800B, in various embodiments, includes an amount of the compound 2802 including silver particles in the range of about 0.1 ounces to about 1 ounce per pound, by weight and/or volume. In certain embodiments, the medicated starter food 2800B includes about 0.5 ounces of the compound 2802 including silver particles per pound, by weight and/or volume.

The method 3900 further includes facilitating daily ingestion of the silver particles by the juvenile animal (block 3904). Facilitating daily ingestion of the silver particles by the juvenile animal may include feeding the medicated starter food 2800B to the juvenile animal. The medicated starter food 2800B may be fed to the juvenile animal in any suitable technique and/or method that encourages and/or causes a juvenile animal to ingest the silver particles on a daily and/or other similar regular basis. Examples of providing the silver compound to the juvenile animal can include, but is not limited to, automatically and/or passively (e.g., via regular feeding) and/or manual administration by a caregiver (e.g., via force feeding, etc.), among other techniques/methods that are possible, each of which is contemplated herein.

In various embodiments, providing the medicated starter food 2800B and facilitating daily ingestion of the silver particles is performed to kill the bacteria Clostridium perfringens in the digestive system (e.g., intestines) of the juvenile animal, and juvenile fowl in particular. In additional or alternative embodiments, providing the medicated starter food 2800B and facilitating daily ingestion of the silver particles is performed to prevent and/or treat necrotic enteritis in the juvenile animal, and especially in juvenile fowl.

In various embodiments, facilitating daily ingestion or causing daily ingestion of the silver particles (e.g., via manual administration) may include ingestion of the silver particles any suitable number of times a day. For example, ingesting an amount of the silver particles can include ingestion once a day, two times a day, three times a day, etc., among other quantities that are possible, each of which is contemplated herein. In certain embodiments, facilitating daily ingestion or causing daily ingestion of the silver particles may include the compound including the silver particles being added to each feed 2804B present to the animal (e.g., sprayed on the feed 2804B one or more times a day).

In at least some embodiments in which the silver particles are ingested or caused to be ingested at least twice daily, each ingestion is separated by a predetermined minimum amount of time, which can be any suitable amount of time that can facilitate protecting and/or treating the digestive system of an animal. Example predetermined minimum amounts of time can include, but are not limited to, one hour, two hours, four hours, six hours, eight hours, twelve hours, etc., among other minimum amounts of time that are possible, each of which is contemplated herein.

Block 3904 can be performed while the animal is a juvenile, which can be any amount of time, as discussed else wherein. In certain embodiments, the juvenile animal is provided the medicated starter food 2800B for 17 days, among other amounts of time greater than or less than 17 days that are possible, each of which is contemplated herein.

After reaching adulthood, a non-medicated feed (e.g., feed 2804C) is provided to the animal (block 3906). The non-medicated feed (e.g., feed 2804C) does not include Ag particles and is provided to the animal for the rest of its life.

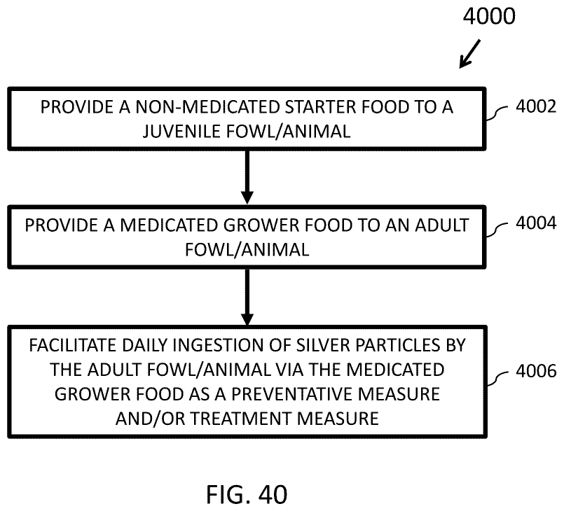

FIG. 40 is a block diagram of one embodiment of a method 4000 for using a non-medicated feed (e.g., feed 2804B) when an animal is a juvenile and using a medicated grower food (e.g., medicated grower food 2800C) to protect the digestive system of an adult animal (e.g., fowl, livestock, or other animal) from the bacteria Clostridium perfringens, prevent necrotic enteritis in the adult animal, and/or treat necrotic enteritis in the adult animal. At least in the illustrated embodiment, a non-medicated feed (e.g., feed 2804B) is provided to an animal when the animal is a juvenile (block 4002).

Block 4002 can be performed while the animal is a juvenile, which can be any amount of time, as discussed else wherein. In certain embodiments, the juvenile animal is provided the non-medicated feed (e.g., feed 2804B) for 17 days, among other amounts of time greater than or less than 17 days that are possible, each of which is contemplated herein.

The method 4000 further include providing a medicated grower food 2800C to the animal upon reaching adulthood (block 4004). The medicated grower food 2800C may include any embodiment of the medicated grower food 2800C discussed elsewhere herein.

The medicated grower food 2800C can be provided to the adult animal in any suitable manner that is known or developed in the future. Examples of providing the medicated grower food 2800C to the adult animal can include, but is not limited to, automatically and/or passively (e.g., via food trough) and/or manual administration by a caregiver (e.g., via force feeding, etc.), among other techniques/methods that are possible, each of which is contemplated herein.

The silver particles in the compound 2802 can include any embodiment of the silver particles discussed herein. That is, the silver particles may include nano-silver particles 100, colloidal silver particles, ionic silver particles, solution silver particles, and/or suspension silver particles.

In some embodiments, the compound 2802 includes an amount of silver particles (e.g., Ag particles), which can include any amount of silver particles in the range of about 0.5 ppm to about 100 ppm, by weight and/or volume, as discussed elsewhere herein. In certain embodiments, the amount of silver particles is 10 ppm or 32 ppm, by weight and/or volume, among other amounts that are possible, each of which is contemplated herein.

In various embodiments, the ratio of compound 2802 including silver particles and feed 2804C is in the range of about 1:50 to about 1:10, as discussed elsewhere herein. In certain embodiments, the ratio of compound 2802 including silver particles and feed 2804C is about 1:30, as discussed elsewhere herein.

The medicated grower food 2800C, in various embodiments, includes an amount of the compound 2802 including silver particles in the range of about 0.1 ounces to about 1 ounce per pound, by weight and/or volume. In certain embodiments, the medicated starter food 2800C includes about 0.5 ounces of the compound 2802 including silver particles per pound, by weight and/or volume.

The method 4000 further includes facilitating daily ingestion of the silver particles by the adult animal (block 4006). Facilitating daily ingestion of the silver particles by the adult animal may include feeding the medicated grower food 2800C to the adult animal. The medicated grower food 2800C may be fed to the adult animal in any suitable technique and/or method that encourages and/or causes an adult animal to ingest the silver particles on a daily and/or other similar regular basis. Examples of providing the silver compound to the adult animal can include, but is not limited to, automatically and/or passively (e.g., via regular feeding) and/or manual administration by a caregiver (e.g., via force feeding, etc.), among other techniques/methods that are possible, each of which is contemplated herein.

In various embodiments, providing the medicated grower food 2800C and facilitating daily ingestion of the silver particles is performed to kill the bacteria Clostridium perfringens in the digestive system (e.g., intestines) of the adult animal, and adult fowl in particular. In additional or alternative embodiments, providing the medicated grower food 2800C and facilitating daily ingestion of the silver particles is performed to prevent and/or treat necrotic enteritis in the adult animal, and especially in adult fowl.

In various embodiments, facilitating daily ingestion or causing daily ingestion of the silver particles (e.g., via manual administration) may include ingestion of the silver particles any suitable number of times a day. For example, ingesting an amount of the silver particles can include ingestion once a day, two times a day, three times a day, etc., among other quantities that are possible, each of which is contemplated herein. In certain embodiments, facilitating daily ingestion or causing daily ingestion of the silver particles may include the compound including the silver particles being added to each feed 2804C present to the animal (e.g., sprayed on the feed 2804C one or more times a day).

In at least some embodiments in which the silver particles are ingested or caused to be ingested at least twice daily, each ingestion is separated by a predetermined minimum amount of time, which can be any suitable amount of time that can facilitate protecting and/or treating the digestive system of an animal. Example predetermined minimum amounts of time can include, but are not limited to, one hour, two hours, four hours, six hours, eight hours, twelve hours, etc., among other minimum amounts of time that are possible, each of which is contemplated herein.

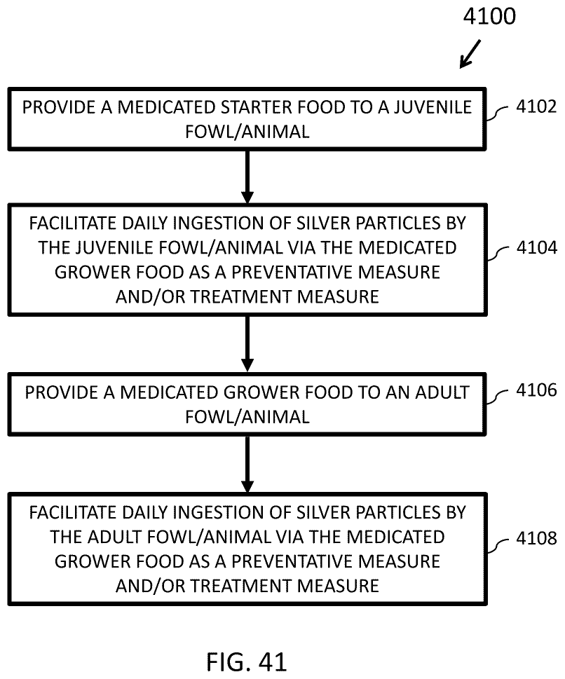

FIG. 41 is a block diagram of one embodiment of a method 4100 for using a medicated starter food (e.g., medicated starter food 2800B) to protect the digestive system of a juvenile animal (e.g., fowl, livestock, or other animal) from the bacteria Clostridium perfringens, prevent necrotic enteritis in the juvenile animal, and/or treat necrotic enteritis in the juvenile animal and using a medicated grower food (e.g., medicated grower food 2800C) to protect the digestive system of the animal from the bacteria Clostridium perfringens, prevent necrotic enteritis in the animal, and/or treat necrotic enteritis in the animal when the animal reaches adulthood. At least in the illustrated embodiment, the method 4100 can begin by providing a medicated starter food 2800B to a juvenile animal (block 4102). The medicated starter food 2800B may include any embodiment of the medicated starter food 2800B discussed elsewhere herein.

The medicated starter food 2800B can be provided to the juvenile animal in any suitable manner that is known or developed in the future. Examples of providing the medicated starter food 2800B to the juvenile animal can include, but is not limited to, automatically and/or passively (e.g., via food trough) and/or manual administration by a caregiver (e.g., via force feeding, etc.), among other techniques/methods that are possible, each of which is contemplated herein.

The silver particles in the compound 2802 can include any embodiment of the silver particles discussed herein. That is, the silver particles may include nano-silver particles 100, colloidal silver particles, ionic silver particles, solution silver particles, and/or suspension silver particles.

In some embodiments, the compound 2802 includes an amount of silver particles (e.g., Ag particles), which can include any amount of silver particles in the range of about 0.5 ppm to about 100 ppm, by weight and/or volume, as discussed elsewhere herein. In certain embodiments, the amount of silver particles is 10 ppm or 32 ppm, by weight and/or volume, among other amounts that are possible, each of which is contemplated herein.

In various embodiments, the ratio of compound 2802 including silver particles and feed 2804B is in the range of about 1:50 to about 1:10, as discussed elsewhere herein. In certain embodiments, the ratio of compound 2802 including silver particles and feed 2804B is about 1:30, as discussed elsewhere herein.

The medicated starter food 2800B, in various embodiments, includes an amount of the compound 2802 including silver particles in the range of about 0.1 ounces to about 1 ounce per pound, by weight and/or volume. In certain embodiments, the medicated starter food 2800B includes about 0.5 ounces of the compound 2802 including silver particles per pound, by weight and/or volume.

The method 4100 further includes facilitating daily ingestion of the silver particles by the juvenile animal (block 4104). Facilitating daily ingestion of the silver particles by the juvenile animal may include feeding the medicated starter food 2800B to the juvenile animal. The medicated starter food 2800B may be fed to the juvenile animal in any suitable technique and/or method that encourages and/or causes a juvenile animal to ingest the silver particles on a daily and/or other similar regular basis. Examples of providing the silver compound to the juvenile animal can include, but is not limited to, automatically and/or passively (e.g., via regular feeding) and/or manual administration by a caregiver (e.g., via force feeding, etc.), among other techniques/methods that are possible, each of which is contemplated herein.

In various embodiments, providing the medicated starter food 2800B and facilitating daily ingestion of the silver particles is performed to kill the bacteria Clostridium perfringens in the digestive system (e.g., intestines) of the juvenile animal, and juvenile fowl in particular. In additional or alternative embodiments, providing the medicated starter food 2800B and facilitating daily ingestion of the silver particles is performed to prevent and/or treat necrotic enteritis in the juvenile animal, and especially in juvenile fowl.

In various embodiments, facilitating daily ingestion or causing daily ingestion of the silver particles (e.g., via manual administration) may include ingestion of the silver particles any suitable number of times a day. For example, ingesting an amount of the silver particles can include ingestion once a day, two times a day, three times a day, etc., among other quantities that are possible, each of which is contemplated herein. In certain embodiments, facilitating daily ingestion or causing daily ingestion of the silver particles may include the compound including the silver particles being added to each feed 2804B present to the animal (e.g., sprayed on the feed 2804B one or more times a day).

In at least some embodiments in which the silver particles are ingested or caused to be ingested at least twice daily, each ingestion is separated by a predetermined minimum amount of time, which can be any suitable amount of time that can facilitate protecting and/or treating the digestive system of an animal. Example predetermined minimum amounts of time can include, but are not limited to, one hour, two hours, four hours, six hours, eight hours, twelve hours, etc., among other minimum amounts of time that are possible, each of which is contemplated herein.

Block 4104 can be performed while the animal is a juvenile, which can be any amount of time, as discussed else wherein. In certain embodiments, the juvenile animal is provided the medicated starter food 2800B for 17 days, among other amounts of time greater than or less than 17 days that are possible, each of which is contemplated herein.

The method 4100 further include providing a medicated grower food 2800C to the animal upon reaching adulthood (block 4106). The medicated grower food 2800C may include any embodiment of the medicated grower food 2800C discussed elsewhere herein.

The medicated grower food 2800C can be provided to the adult animal in any suitable manner that is known or developed in the future. Examples of providing the medicated grower food 2800C to the adult animal can include, but is not limited to, automatically and/or passively (e.g., via food trough) and/or manual administration by a caregiver (e.g., via force feeding, etc.), among other techniques/methods that are possible, each of which is contemplated herein.

The silver particles in the compound 2802 can include any embodiment of the silver particles discussed herein. That is, the silver particles may include nano-silver particles 100, colloidal silver particles, ionic silver particles, solution silver particles, and/or suspension silver particles.

In some embodiments, the compound 2802 includes an amount of silver particles (e.g., Ag particles), which can include any amount of silver particles in the range of about 0.5 ppm to about 100 ppm, by weight and/or volume, as discussed elsewhere herein. In certain embodiments, the amount of silver particles is 10 ppm or 32 ppm, by weight and/or volume, among other amounts that are possible, each of which is contemplated herein.

In various embodiments, the ratio of compound 2802 including silver particles and feed 2804C is in the range of about 1:50 to about 1:10, as discussed elsewhere herein. In certain embodiments, the ratio of compound 2802 including silver particles and feed 2804C is about 1:30, as discussed elsewhere herein.

The medicated grower food 2800C, in various embodiments, includes an amount of the compound 2802 including silver particles in the range of about 0.1 ounces to about 1 ounce per pound, by weight and/or volume. In certain embodiments, the medicated starter food 2800C includes about 0.5 ounces of the compound 2802 including silver particles per pound, by weight and/or volume.

The method 4100 further includes facilitating daily ingestion of the silver particles by the adult animal (block 4108). Facilitating daily ingestion of the silver particles by the adult animal may include feeding the medicated grower food 2800C to the adult animal. The medicated grower food 2800C may be fed to the adult animal in any suitable technique and/or method that encourages and/or causes an adult animal to ingest the silver particles on a daily and/or other similar regular basis. Examples of providing the silver compound to the adult animal can include, but is not limited to, automatically and/or passively (e.g., via regular feeding) and/or manual administration by a caregiver (e.g., via force feeding, etc.), among other techniques/methods that are possible, each of which is contemplated herein.

In various embodiments, providing the medicated grower food 2800C and facilitating daily ingestion of the silver particles is performed to kill the bacteria Clostridium perfringens in the digestive system (e.g., intestines) of the adult animal, and adult fowl in particular. In additional or alternative embodiments, providing the medicated grower food 2800C and facilitating daily ingestion of the silver particles is performed to prevent and/or treat necrotic enteritis in the adult animal, and especially in adult fowl.

In various embodiments, facilitating daily ingestion or causing daily ingestion of the silver particles (e.g., via manual administration) may include ingestion of the silver particles any suitable number of times a day. For example, ingesting an amount of the silver particles can include ingestion once a day, two times a day, three times a day, etc., among other quantities that are possible, each of which is contemplated herein. In certain embodiments, facilitating daily ingestion or causing daily ingestion of the silver particles may include the compound including the silver particles being added to each feed 2804C present to the animal (e.g., sprayed on the feed 2804C one or more times a day).

In at least some embodiments in which the silver particles are ingested or caused to be ingested at least twice daily, each ingestion is separated by a predetermined minimum amount of time, which can be any suitable amount of time that can facilitate protecting and/or treating the digestive system of an animal. Example predetermined minimum amounts of time can include, but are not limited to, one hour, two hours, four hours, six hours, eight hours, twelve hours, etc., among other minimum amounts of time that are possible, each of which is contemplated herein.

In some embodiments, the medicated starter food 2800B and the medicated grower food 2800C include the same amount and/or ratio of the silver compound to feed 2804B/2804C. In other embodiments, the medicated starter food 2800B and the medicated grower food 2800C include different amounts and/or ratios of the silver compound to feed 2804B/2804C. In certain embodiments, the medicated starter food 2800B includes a higher amount and/or ratio of the silver compound to feed 2804B than the medicated grower food 2800C. In other embodiments, the medicated grower food 2800C includes a higher amount and/or ratio of the silver compound to feed 2804C than the medicated starter food 2800B.

Aspects of the various embodiments discussed above may be embodied as a compound and/or method for using silver compounds to protect the digestive system, prevent digestive system disease, and/or treat disease in the digestive system of a fowl (or other livestock animal). Other aspects of the various embodiments discussed above may be embodied as a compound and/or method for using silver compounds to protect the respiratory system, prevent respiratory system disease, and/or treat disease in the digestive system of an animal (e.g., a livestock animal or other animal). Reference throughout this specification to one embodiment, an embodiment, or similar language means that a particular feature, structure, or characteristic described in connection with the embodiment is included in at least one embodiment. Thus, appearances of the phrases in one embodiment, in an embodiment, and similar language throughout this specification may, but do not necessarily, all refer to the same embodiment, but mean one or more but not all embodiments unless expressly specified otherwise. The terms including, comprising, having, and variations thereof mean including but not limited to, unless expressly specified otherwise. An enumerated listing of items does not imply that any or all of the items are mutually exclusive, unless expressly specified otherwise. The terms, “a,” “an,” and “the,” also refer to one or more unless expressly specified otherwise.

In addition, as used herein, the term, “set,” can mean one or more, unless expressly specified otherwise. The term, “sets,” can mean multiples of or a plurality of one or mores, ones or more, and/or ones or mores consistent with set theory, unless expressly specified otherwise.

Furthermore, the described features, structures, or characteristics of the embodiments may be combined in any suitable manner. One skilled in the relevant art will recognize, however, that embodiments may be practiced without one or more of the specific details, or with other methods, components, materials, and so forth. In other instances, well-known structures, materials, or operations are not shown or described in detail to avoid obscuring aspects of an embodiment.

Aspects of the embodiments are described above with reference to schematic flowchart diagrams and/or schematic block diagrams of particles (e.g., silver particles 100) and methods according to various embodiments. It should also be noted that, in some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. Other steps and methods may be conceived that are equivalent in function, logic, or effect to one or more blocks, or portions thereof, of the illustrated Figures.

Although various arrow types and line types may be employed in the flowchart and/or block diagrams, they are understood not to limit the scope of the corresponding embodiments. Indeed, some arrows or other connectors may be used to indicate only the logical flow of the depicted embodiment. For instance, an arrow may indicate a waiting or monitoring period of unspecified duration between enumerated steps of the depicted embodiment.

The description of elements in each figure may refer to elements of proceeding figures. Like numbers refer to like elements in all figures, including alternate embodiments of like elements.

The various disclosed embodiments may be practiced in other specific forms. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A method, comprising:
providing a compound comprising silver (Ag) particles; and
facilitating daily oral ingestion of the compound by an animal,
wherein providing the compound and facilitating daily oral ingestion of the compound by the animal is performed to protect the digestive system of the animal.

2. The method of claim 1, wherein:
providing the compound comprising Ag particles to the animal comprises providing a medicated food to the animal, the medicated food comprising the Ag particles and feed; and
facilitating daily oral ingestion of the compound comprises facilitating daily ingestion of the Ag particles via the medicated food.

3. The method of claim 2, wherein the animal includes a fowl.

4. The method of claim 2, wherein the medicated food comprises 0.1 ounces to 1 ounce of the Ag particles per pound.

5. The method of claim 4, wherein the Ag particles comprise one of nano-silver particles and colloidal silver particles.

6. The method of claim 2, wherein the medicated food comprises a ratio of Ag particles to feed in the range of about 1:10 to about 1:50.

7. The method of claim 6, wherein the Ag particles comprise one of nano-silver particles and colloidal silver particles.

8. The method of claim 2, wherein facilitating daily ingestion of the Ag particles by the animal comprises facilitating daily ingestion of at least 4 ounces of the medicated food.

9. The method of claim 2, wherein facilitating daily ingestion of the Ag particles by the animal comprises facilitating ingestion of the medicated food two times daily.

10. The method of claim 9, wherein facilitating daily ingestion of the compound two times daily comprises facilitating ingestion of 2 ounces of the medicated food two times daily.

11. The method of claim 2, wherein providing the medicated food to the animal and facilitating daily ingestion of the Ag particles by the animal medicated food is performed to prevent necrotic enteritis.

12. The method of claim 11, wherein providing the medicated food to the animal and facilitating daily ingestion of the Ag particles by the animal via the medicated food is performed to kill Clostridium perfringens.

13. The method of claim 2, wherein providing the medicated food to the animal and facilitating daily ingestion of the Ag particles by the animal via the medicated food is performed to kill Clostridium perfringens.

14. A method, comprising:
facilitating daily oral ingestion of a first compound comprising silver (Ag) particles via a medicated starter food by a juvenile animal; and
in response to the juvenile animal maturing into an adult animal, facilitating daily oral ingestion of a second compound comprising Ag particles via a medicated grower food.

15. The method of claim 14, wherein the first compound comprising Ag particles in the medicated starter food and the second compound comprising Ag particles in the medicated grower food include one of a same amount of Ag particles by weight or volume or different amounts of Ag particles by weight or volume.

16. A food for at least one of treating or preventing necrotic enteritis in an animal, comprising:
a feed; and
a compound comprising silver (Ag) particles combined with the feed to create a medicated food for the animal,
wherein the medicated food is configured to protect the digestive system of the animal.

17. The food of claim 16, wherein the compound comprising Ag particles comprises 0.5 ppm to 100 ppm of the Ag particles.

18. The food of claim 16, wherein the Ag particles comprise one of nano-silver particles and colloidal silver particles.

19. The food of claim 16, wherein the medicated food comprises a ratio of Ag particles to feed in the range of 1:50 to 1:10.

20. The food of claim 16, wherein the medicated food comprises 0.1 ounces to 1.0 ounce of the Ag particles by weight per pound of the medicated food.
